(19) United States
(12) Patent Application Publication
(10) Pub. No.: US 2015/0126597 A1
(43) Pub. Date: May 7, 2015

(54) GENISTEIN CANCER TREATMENT REGIMEN MAXIMIZING CANCER RADIATION THERAPY BENEFITS

(71) Applicant: Humanetics Corporation, Minneapolis, MN (US)

(72) Inventors: Michael D. Kaytor, Maplewood, MN (US); John L. Zenk, Eden Prairie, MN (US)

(21) Appl. No.: 14/074,468

(22) Filed: Nov. 7, 2013

(51) Int. Cl. A61K 31/352

(52) U.S. Cl. CPC A61K 31/352
USPC 514/456

(57) ABSTRACT

A treatment regimen for a patient undergoing fractionated radiation therapy for a solid tumor type cancer to attain an improvement in one or more of and an optimized balancing of cancer cell radiation sensitization, normal cell radioprotection, selective post-exposure inhibition of cancer cell recovery relative to normal cell recovery, and stabilization of body weight during radiation treatment. The regimen comprises administration of a therapeutic amount of genistein to a patient diagnosed with a solid tumor type cancer throughout an administration period that commences at least five days prior to commencement of fractionated cancer radiation therapy and extends until at least the conclusion of fractionated cancer radiation therapy.

### GOVERNMENT SUPPORT

This invention was made with government support under Grant Number HHSO100201100026C awarded by the U.S. Department of Health and Human Services, Biomedical Advanced Research and Development Authority. The government has certain rights in this invention.

This invention was made with government support under Grant Number HHSN261201200078C awarded by the US Department of Health and Human Services, National Cancer Institute. The government has certain rights in this invention.

### FIELD OF INVENTION

The invention relates to methods of improving the effectiveness of radiation therapy for solid type cancers without a concomitant increase in radiative damage of surrounding normal cells.

### BACKGROUND

The two major types of lung cancer are known as small cell lung cancer (SCLC) and non-small cell lung cancer (NSCLC). NSCLC constitutes about 85% to 90% of lung cancers. Lung cancer (both SCLC and NSCLC) is the second most common cancer in the United States. Lung cancer accounts for about 14% of all new cancers. The American Cancer Society estimates that nearly 230,000 new cases of lung cancer will be diagnosed in the United States in 2013.

The chance that a man will develop lung cancer in his lifetime is about 1 in 13. For woman it is about 1 in 16. These numbers include both smokers and non-smokers, with the risk much higher for smokers than non-smokers.

There are three main subtypes of NSCLC. Adenocarcinoma accounts for about 40% of lung cancers. These cancers start as progenitor cells that would normally secrete substances such as mucus. This type of lung cancer occurs mainly in current or former smokers, but it is also the most common type of lung cancer seen in non-smokers. It is more common in women than in men, and it is more likely to occur in younger people than other types of lung cancer. Adenocarcinoma is usually found in outer parts of the lung. It tends to grow slower than other types of lung cancer, and is more likely to be found before it has spread outside of the lung. Squamous cell (epidermoid) carcinoma accounts for about 25% to 30% of all lung cancers. These cancers start as progenitors of squamous cells, which are flat cells that line the inside of the airways in the lungs. They are often linked to a history of smoking and tend to be found in the middle of the lungs, near a bronchus. Large cell (undifferentiated) carcinoma accounts for about 10% to 15% of lung cancers. It can appear in any part of the lung. It tends to grow and spread quickly, which can make it harder to treat. There are also a few other subtypes of NSCLC, such as adenosquamous carcinoma and sarcomatoid carcinoma, but these types are much less common.

Treatment for NSCLC commonly includes radiation therapy. Radiation therapy uses high-energy rays (such as x-rays) or particles to kill cancer cells. There are two main types of radiation therapy—external beam radiation therapy (EBRT) and brachytherapy (internal radiation therapy), with EBRT employed far more than brachytherapy in the treatment of lung cancer. EBRT focuses radiation from outside the body on the cancer. Most often EBRT radiation treatment is fractionated (e.g., given 5 days a week for 5 to 7 weeks). EBRT has been refined in recent years to include three-dimensional conformal radiation therapy (3D-CRT), intensity modulated radiation therapy (IMRT) and stereotactic body radiation therapy (SBRT), each of which rely upon special computers to precisely map the location of the tumor(s) and control delivery of radiation.

Common side effects with radiation therapy include, sunburn-like skin problems, hair loss where the radiation enters the body, fatigue, nausea and vomiting, loss of appetite and weight loss.

Significant advances have been made over the years in the diagnosis and treatment of lung cancer, but it remains the leading cause of cancer death among both men and women by a large margin. The five year survival rate for persons diagnosed with lung cancer is between about one-half to one-third when the cancer is diagnosed and treated early, with a precipitous drop when diagnosed in the mid and later stages.

Hence, a substantial need continues to exist for a method of improving the effectiveness of radiation therapy for solid type cancers such as NSCLC, preferably without a concomitant increase in the severity of associated side effects.

### SUMMARY OF THE INVENTION

The invention is directed to a treatment regimen for a patient undergoing fractionated radiation therapy for a solid tumor type cancer. The regimen comprises administration of a therapeutic amount of genistein to a patient diagnosed with a solid tumor type cancer throughout an administration period that commences at least five days prior to commencement of fractionated cancer radiation therapy and extends until at least the conclusion of fractionated cancer radiation therapy.

In a preferred embodiment, the treatment regimen attains an improvement in one or more of cancer cell radiation sensitization, normal cell radioprotection, selective post-exposure inhibition of cancer cell recover relative to normal cell recovery, and/or stabilization of body weight during radiation treatment, by administering a standard daily therapeutic amount of genistein to the patient throughout an administration period that commences at least one week prior to commencement of fractionated cancer radiation therapy employing fractionated doses of radiation between about 1.5 and 2.5 Gy per dose, and extends until at least the conclusion of fractionated cancer radiation therapy. The treatment regimen preferably attains an optimal balancing of cancer cell radiation sensitization, normal cell radioprotection, and selective post-exposure inhibition of cancer cell recover relative to normal cell recovery.

### BRIEF DESCRIPTION OF THE FIGURES

FIGS. 1A, 1B and 1C are plots of Normalized Tumor Volume v. Days Post IR observed and measured in Example One. Tumor growth characteristics following ten weeks of BIO 300 dosing with or without a single dose (12.5 Gy) of tumor irradiation. Data were normalized and plotted with day 0 being within 48 hours of tumor irradiation (mean±SEM). One-way ANOVA was used with Dunnett's multiple comparisons post-hoc analysis. Compared to no treatment; *p<0.05; **p<0.01.

FIGS. 2A, 2B and 2C are plots of Normalized Weight v. Days measured in Example One. Body weights were collected twice weekly, normalized, and plotted versus time on study (mean±SEM). One-way ANOVA was used followed by Dunnett's multiple comparisons post-hoc tests to analyze the data. Compared to the vehicle only group; *p<0.05.

FIGS. 3A and 3B are plots of wet lung weights measured in Example One. The weights were determined following euthanasia and plotted as individual points with the mean weight and SEM shown. FIG. 3A includes all experimental groups. FIG. 3B includes irradiated animals without A549 xenograft.

FIG. 4 is a set of microscopic images employed in the histopathology analysis of lung tissue collected from animals that received vehicle (with or without radiation) and BIO 300 dosed at 200 mg/kg or 400 mg/kg (both with radiation treatment) in Example 1.

FIG. 5 is a plot of severity scores for observed pathological lung damage in Example 1. Severity scores are arbitrary numbers assigned by the pathologist based on the severity of the tissue damage (scale 0-4).

FIG. 6 is the experimental design for Example 2.

FIGS. 7A and 7B are plots of Normalized Tumor Volume for the different experimental groups v. Days Post IR observed and measured in Example Two. The plot includes only the mice that completed the study as noted in the legend. The Cox regression analysis depicted in 7B indicates that, compared to radiation alone, the addition of BIO 300 (all groups) reduces the risk of the tumor from doubling (p=0.008, HR=0.26, 95% CI: 0.10-0.95).

FIGS. 8A, 8B, 8C and 8D are Cox regression analysis of tumor growth characteristics from Example 2. For this analysis the definition of an event was set as a doubling in tumor volume, the baseline being the tumor volume on the day of radiation. FIG. 8A is BIO 300 (400 mg/kg) as a monotherapy. FIG. 8B compares BIO 300 doses. Compared to radiation alone BIO 300 (200 mg/kg) reduces the risk of the tumor from doubling (p=0.0036, HR=0.10, 95% CI: 0.1-0.47). FIG. 8C depicts BIO 300 (200 mg/kg) dosed 31 days (early) or 7 days (late) prior to radiation treatment. FIG. 8D depicts BIO 300 (400 mg/kg) dosed 31 days (early) or 7 days (late) prior to radiation treatment. When early dosing groups were combined, initiation of BIO 300 dosing early (31 days prior to radiation in this study) was statistically different than radiation alone (p=0.027, HR=0.25, 95% CI: 0.07-0.85). Similarly, when late dosing groups were combined, initiation of BIO 300 dosing late (7 days prior to radiation treatment in this study) was statistically different than radiation alone (p=0.027, HR=0.27, 95% CI: 0.09-0.86).

FIG. 9 correlates calculated tumor volumes and actual physical tumor weight at the time of euthanasia for data obtained in Example 2.

FIGS. 10A, 10B and 10C are plots of body weight v. time on study per treatment group for data obtained in Example 2. Student's t-test was used to compare the mean body weight on each given day of the BIO 300 treatment group to the radiation alone group (Vehicle+IR) (radiation alone). *p<0.05. For these plots the day of radiation is Day 31. FIG. 10A depicts BIO 300 (400 mg/kg) evaluated as a monotherapy. FIG. 10B depicts BIO 300 dosed early (31 days prior to radiation). FIG. 10C depicts BIO 300 dosed late (7 days prior to radiation).

FIGS. 11A and 11B are plots of GI tract and uterus wet tissue weights at the time of euthanasia, normalized to animal body weight and plotted as individual points with the mean shown by the horizontal bar. FIG. 11A is normalized uterus weights. FIG. 11B is normalized GI tract weights.

FIG. 12 is a plot of wet lung weight measured in Example Three among sham-IR, IR Only, IR+BIO300, and IR+Vehicle. At the time of necropsy, wet lung weights are recorded as a surrogate marker for pulmonary edema and congestion. * p<0.05 IR+BIO 300 vs. WTLI alone. Error bars represent ±SEM.

FIG. 13 is a graph depicting Percent reduction in fibrosis measured in Example Three between BIO 300 treated animals versus radiation alone.

FIG. 14 is a set of microscopic images employed in the histopathology analysis of lung tissue collected 180 days post radiation from animals that received sham radiation, radiation, and BIO 300 with radiation in Example 3.

### DETAILED DESCRIPTION OF A PREFERRED EMBODIMENT

### Theory

Without intending to be restricted thereby, it is believed that radiation sensitization of cancer cells results from cell cycle arrest and induction of apoptotic pathways.

Without intending to be restricted thereby, it is believed that the protection of normal lung tissue results from a reduction in congestion (pneumonitis), a mitigation of the influx of inflammatory cells, an increase in free radical scavenging, and a dampening of radiation-induced pulmonary fibrosis.

Without intending to be restricted thereby, it is believed that the selective post-exposure inhibition of cancer cell recovery relative to normal cell recovery is mediated by the different proliferative states of cancer and normal cells.

Without intending to be restricted thereby, it is believed that stabilization of body weight during radiation treatment results from an increased rate of weight gain prior to radiation treatment.

### Description

Administration of a therapeutically effective dosage of genistein to a patient undergoing fractionated radiation therapy for a solid tumor type cancer throughout an administration period that commences at least five days prior to commencement of fractionated cancer radiation therapy employing fractionated doses of radiation between about 1.5 and 2.5 Gy per dose, and extends until at least the conclusion of fractionated cancer radiation therapy, can attain an improvement in one or more of cancer cell radiation sensitization, normal cell radioprotection, and selective post-exposure inhibition of cancer cell recovery relative to normal cell recovery (collectively “tripartite therapy enhancements”). The treatment regimen can attain an optimal balancing of the tripartite therapy enhancements, and even attain an improvement in each aspect of the tripartite therapy enhancements. The treatment regimen can also stabilize body weight during radiation treatment.

Indications

The treatment regimen of this invention can be beneficially employed with and is indicated in those instances where radiation therapy is employed to treat a solid type tumor, including specifically but not exclusively sarcomas, carcinomas and lymphomas such as bladder cancer, breast cancer, colon and rectal cancer, endometrial cancer, kidney (renal cell) cancer, lung cancer including both small cell lung cancer (SCLC) and non-small cell lung cancer (NSCLC), melanoma, non-hodgkin lymphoma, pancreatic cancer, prostate cancer and thyroid cancer. Of particular interest is implementation of the treatment regimen in connection with radiation treatment for NSCLC, due in part to the strong need for both radiation sensitization of the cancer cells and radioprotection of the surrounding normal cells as radiation levels generally necessary to effectively treat this type of cancer results in significant collateral damage to the surrounding normal lung tissue.

Active Agent

Genistein belongs to the pharmacological classes of soy isoflavone, flavonoid, polyphenol and phytoestrogen. It is also known as 5,7-dihydroxy-3-(4-hydroxyphenyl)-chromen-4-one (IUPAC), 5,7-dihydroxy-3-(4-hydroxyphenyl)-4H-1-benzopyran-4-one, 5,7,4′-trihydroxyisoflavone, 4′,5,7-trihydroxyisoflavone, Genestein, Prunetol, Sophoricol and Differenol A. It has a Molecular Formula of C₁₅H₁₀O₅, a Molecular Weight of 270.237 g/mol (270.24 daltons), a Chemical Abstracts Service (CAS) Registry Number 446-72-0 and a Beilstein Registry Number 263823. It is commercially available from a number of sources, including DSM Nutritional Products, Inc. of Basel, Switzerland under Drug Master File (DMF) #19747 and further described in PIND #119322.

Administration

Administration Route

Genistein can be administered by virtually any of the commonly accepted practices for the administration of pharmaceutical preparations including specifically, but not exclusively, mucosal administration, oral consumption, ocular administration, subcutaneous injection, transdermal administration, intravascular administration, intramuscular administration, etc. Oral administration is generally preferred.

Mucosal administration of genistein includes such routes as buccal, endotracheal, nasal, pharyngeal, rectal, sublingual, vaginal, etc. For administration through the buccal/sublingual/pharyngeal/endotracheal mucosal, genistein may be formulated as an emulsion, gum, lozenge, spray, tablet or an inclusion complex such as cyclodextrin inclusion complexes. Nasal administration is conveniently conducted through the use of a sniffing powder or nasal spray. For rectal and vaginal administration, genistein may be formulated as a cream, douche, enema or suppository.

Oral consumption of genistein may be effected by incorporating the genistein into a food or drink, or formulating the genistein into a chewable or swallowable tablet or capsule. The genistein is preferably orally administered as a nanosuspension in accordance with US Patent Application Publications 2012/0164190 and 2012/0121654, both hereby incorporated by reference.

Genistein is virtually insoluble in water, thereby limiting its bioavailability when administered orally. Genistein provided as a nano suspension in accordance with US Patent Application Publications 2012/0164190 and 2012/0121654 has significantly improved oral bioavailability. This allows dosing without medical supervision, which enables pre-dosing at home prior to known and planned instances of radiation therapy. To further improve oral bioavailability, genistein can also be incorporated as sub-micron size particles in an orally ingestible formulation. Generally, a dose of ˜1 g per day of genistein provided as a nanosuspension should be effective for achieving the desired mitigating protective effect.

Ocular administration may be effected by incorporating genistein into a solution or suspension adapted for ocular application such as drops or sprays.

Subcutaneous, intravascular and intramuscular administration involves incorporating the genistein into a pharmaceutically acceptable and injectable carrier.

For transdermal administration, the genistein may be conveniently incorporated into a lipophilic carrier and formulated as a topical cream or adhesive patch.

Administration Dosage and Timing

The range of dosages effective for achieving an improvement in at least one of the tripartite therapy enhancements, preferably including an optimized balancing of the tripartite therapy enhancements, and most preferably an improvement in each of the tripartite therapy enhancements, may be determined in accordance with standard industry practices. The desired tripartite therapy enhancements can generally be achieved by administration of at least ˜1 gram of genistein per day, preferably at least ˜1.2 grams of genistein per day and most preferably at least ˜1.5 grams of genistein per day, taken as a single dose or multiple doses each day. Lower amounts may also be therapeutic.

Genistein administered at least five days prior to commencement of fractionated radiation therapy for lung cancer, throughout such therapy and optionally for a short-term duration after conclusion of such therapy is effective for achieving the desired improvement in the tripartite therapy enhancements. Long-term continued administration of a therapeutic amount of genistein after conclusion of radiation therapy has been found to contribute little towards improvement of any aspect of the tripartite therapy enhancements and is therefore discouraged as unnecessary.

Administration Period

The administration of genistein should commence at least five days prior to commencement of radiation therapy for a solid tumor type cancer, with a preference for commencement at least one week, preferably at least two weeks and most preferably at least four weeks, prior to commencement of radiation therapy. Administration should be continued at least through radiation therapy. Again, long-term continued administration of a therapeutic amount of genistein after conclusion of radiation therapy has been found to contribute little towards improvement of any aspect of the tripartite therapy enhancements and is therefore discouraged as unnecessary.

A reduced maintenance amount of genistein may be administered for a period after completion of the primary administration of genistein. The reduction may be in the form of a reduced dosage (e.g., reduced to less than 60% the amount administered during the therapeutic stage) and/or a reduced frequency (e.g., ½ or ¼ the frequency during the therapeutic stage). When employed, the maintenance period should last for at least one month, preferably at least three months and most preferably at least six months. Shorter durations tend to diminish the benefit obtained by administration of a maintenance dosage, while administration of some maintenance amount of genistein can perpetually benefit the patient.

Elevated Administration Periods

The desired tripartite therapy enhancements can be further improved by punctuating administration of the standard daily therapeutic amount of genistein with administration of an elevated daily amount of genistein timed to correspond with at least some, preferably each and every, exposure of the patient to a dose of radiation. The elevated daily amount of genistein is preferably between about 1.2 to 4 times, most preferably between about 1.5 to 2 times, the standard daily therapeutic amount of genistein, commencing at least 12 hours prior, preferably between 24 and 96 hours prior and most preferably between 24 and 72 hours prior, to exposure of the patient to a dose of radiation, and concludes within 24 hours, preferably within 12 hours and most preferably contemporaneously with, exposure of the patient to a dose of radiation.

### EXAMPLES

### Overview of Study Design

The examples were conducted in a mouse xenograft model of NSCLC. The human NSCLC cell line A549 was used to generate the model used for the examples. A549 cells are adenocarcinomic human alveolar basal epithelial cells. The A549 cell line was isolated through the removal and culturing of cancerous lung tissue from a 58-year-old Caucasian male purchased from ATCC, Manassas, Va. Lung adenocarcinoma tumors constitute approximately 50% of all NSCLC tumors. The A549 xenograft model is widely used to evaluate potential new therapeutics. A549 cells were implanted into the subcutaneous space of female athymic nude mice. Following establishment of the tumors the tumor volumes were calculated twice weekly based on the external measurements of the tumor dimensions. At the time tumor volumes were obtained animal weights were determined. Mice were dosed daily (5 or 7 days/week) with BIO 300, described infra, by oral gavage. To correlate tumor volumes with actual tumor weights the tumors were weighed at the time of euthanasia. Finally, at the end of the in-life phase of the example organ weights were collected and tissues were prepared for histopathological analysis.

### Materials and Methods

Animals:

Female athymic CD1 nu/nu mice 6 to 7 week old from Charles River Laboratories were used. Mice were housed four to a cage and provided food and water ad libitum. Mouse chow was phytoestrogen-free (not containing soy), Teklad 2914 rodent chow (Harlan Laboratories, Haslett, Mich.). Rooms were maintained with a 12 hour light/12 hour dark cycle.

BIO 300 Test and Control Articles:

The test article in this study was BIO 300 Oral Suspension (Humanetics Corporation, Minneapolis, Minn.). The Active drug substance in BIO 300 Oral Suspension is unconjugated, and highly pure synthetic genistein manufactured by DSM Pharmaceutical Products, Inc. under Drug Master File (DMF) #19747 (5,7-dihydroxy-3-(4-hydroxyphenyl)-chromen-4-one) (IUPAC). BIO 300 Oral Suspension is a wet-milled nanosuspension containing 325 mg/mL genistein with a d(50) particle-size distribution of less than 0.2 μm, 5% Povidone K17 (w/w), 0.2% Polysorbate 80 (w/w) in 50 mM phosphate buffered saline (61 mM sodium chloride). BIO 300 drug substance (genistein) belongs to the pharmacological class of soy isoflavone/flavonoid; polyphenol; phytoestrogen. The control article (“Vehicle”) was BIO 300 Oral Suspension without the active ingredient, genistein. Vehicle was delivered by oral gavage using the same treatment regimen as described above for the test article.

BIO 300 Dilution and Weight-Based Dosing:

BIO 300 was supplied in 5 mL vials at a concentration of 325 mg/mL. BIO 300 was diluted with drug vehicle such that mice received either 200 mg/kg in volumes ranging from 0.07 to 0.10 mLs depending on animal weight (22 to 32 g) or 400 mg/kg in volumes ranging from 0.14 to 0.20 mLs depending on animal weight (22 to 32 g). Fresh drug dilutions were prepared weekly. BIO 300 was administered by oral gavage daily (5 days/week in Example 1 and 7 days/week in Example 2). Individual animal dosing was determined based on the average body weight of each study group as determined weekly. BIO 300 and vehicle were administered by oral gavage using 20 gauge×1½″ oral animal feeding needles (Cadence, Inc. Cranston, R.I.).

Tumor Volume Calculation:

Tumor dimensions were recorded twice weekly. Tumor volumes were calculated using the formula of an ellipse, (4/3)πR₁×R₂×R₃ where R₃=(R₁+R₂)/2 and R₁, R₂, and R₃ are the orthogonal radii of the tumor. If tumors ulcerated, the animals were euthanized and excluded from the study. Tumor ulceration occurred unpredictably and independently of the tumor size.

Tumor Irradiation:

A radiation dose of 12.5 Gy was delivered to anesthetized mice (60 mg/kg ketamine and 6 mg/kg xylazine, ip) using a 5000 Ci cesium-137 small animal irradiator (Mark I, J. L. Shepherd, San Fernando, Calif.). The dose rate was approximately 3 Gy per minute. The radiation conformed to a 2 cm wide field along the length of the mouse using a commercial collimator. In Example 1, the 2 cm field was centered on the lung. In Example 2, the 2 cm field was centered on the GI tract. Mice were irradiated in groups of 8. Uniformity was maintained by delivering half the dose with the left side of the mouse closest to the source and half the dose with the right side of the mouse closest to the source.

Dosimetry Confirmation of Irradiator:

To confirm the dose given, the radiation dose from the 137Cs irradiator was measured at the start of the example using LiF microcube dosimeters and GAFchromic radiation-sensitive film. The radiation dose to be given to the mice is 12.5Gy. For the 137Cs irradiator, this dose corresponds to an exposure time of approximately 5 minutes (4.68 minutes). The dosimetry analysis confirmed the desired dose; film exposed to the 137Cs source at 12.5Gy had an optical density within 1% of film exposed to the same dose from a clinical irradiator. All mice that received radiation treatment were exposed 1 hour (±5 minutes) after BIO 300 administration.

Organ Weights/Tumor Weights:

At the time of euthanasia organs were harvested and wet tissue weights were measured. In addition to the tissues the weight of the remaining tumor was determined so that the tumor weight can be correlated with the calculated tumor volumes.

Post Mortem Tissue Processing and Staining:

The various tissues were fixed in formalin, then processed and embedded in paraffin. Hematoxylin and eosin (H&E) staining was done on 5 micron sections from each of the tissues.

Immunohistochemical Analyses:

Methods for Ki67 staining and analysis were completed in accordance with the process described in Jenrow, K. A., et al., Combined Atorvastatin and Ramipril Mitigate Radiation-Induced Impairment of Dentate Gyrus Neurogenesis. J Neurooncol, 2011. 101(3): p. 449-56. Briefly, for Ki67 immunohistochemical staining the sections were deparaffinized, re-hydrated and boiled for 10 minutes in a citrate buffer (pH 6.0). After blocking, the sections were incubated for 2 hours in polyclonal anti Ki67 (Lab Vision, Pittsburgh, Pa.) and then labeled with the appropriate Alexa 555 (Life Technologies, Grand Island, N.Y.) secondary antibody.

Statistical Analyses:

Multiple statistical analyses were used to analyze the data presented in this report. Student's t-test was performed when comparisons were made between two treatment groups at a single time point. Repeated measures analysis of covariance (ANCOVA) was used to analyze animal weights over the course of the study. One way ANOVA followed by multiple comparisons tests were also used to analyze data from the different study groups. Cox regression analysis and linear mixed modeling were used to analyze the rate of tumor growth over time and the effects of BIO 300 with or without tumor radiation on tumor growth. All analysis was performed using both the mean as well as individual data within a treatment group. All statistical analyses were performed at alpha=0.05 (significance) and 0.10 (marginal significance) levels.

### Example 1

TABLE ONE

| | | | | A549 | Tumor | |
|---|---|---|---|---|---|---|
| Group | Agent | Dose | Route | Implant | Radiation | BIO 300 Dose Timing |
| 1 | BIO 300 | NA | Oral | NO | YES | 5 d/wk until last dose in implant mice |
| | Vehicle | | Gavage | | | |
| 2 | BIO 300 | NA | Oral | YES | NO | 5 d/wk until tumor weight ≧900 mg |
| | Vehicle | | Gavage | | | |
| 3 | BIO 300 | NA | Oral | YES | YES | 5 d/wk until tumor weight ≧900 mg |
| | Vehicle | | Gavage | | | |
| 4 | BIO 300 | 200 mg/kg | Oral | YES | YES | 5 d/wk until tumor weight ≧900 mg |
| | | | Gavage | | | |
| 5 | BIO 300 | 400 mg/kg | Oral | YES | YES | 5 d/wk until tumor weight ≧900 mg |
| | | | Gavage | | | |
| 6 | BIO 300 | 200 mg/kg | Oral | YES | NO | 5 d/wk until tumor weight ≧900 mg |
| | | | Gavage | | | |
| 7 | BIO 300 | 400 mg/kg | Oral | YES | NO | 5 d/wk until tumor weight ≧900 mg |
| | | | Gavage | | | |
| 8 | BIO 300 | 200 mg/kg | Oral | NO | YES | 5 d/wk until last dose in implant mice |
| | | | Gavage | | | |
| 9 | BIO 300 | 400 mg/kg | Oral | NO | YES | 5 d/wk until last dose in implant mice |
| | | | Gavage | | | |

A549 Xenograft Model and Tumor Location:

A549 cells were seeded within the subcutaneous space in the upper torso approximately 1 cm below the armpit on the dorsal side of the mouse. We choose this location to include the lungs in the radiation field to further assess the radioprotective effects of BIO 300 in normal tissues. The 2 cm wide radiation path used in this example will not only irradiate the tumor but also irradiate the lungs and possibly the upper GI tract. This will allow an assessment of BIO 300 efficacy on the tumor itself, in combination with radiation, and also the radioprotective effects on these surrounding normal tissues. A549 NSCLC cells were implanted into 108 CD1 female athymic nude mice. Of the 108 implanted mice, 104 (96%) developed tumors.

In-Life Study Activities:

2×10⁶ A549 cells were implanted in a 1:1 mixture with Matrigel (100 ul total) at each tumor site. BIO 300 weight based dosing was adjusted weekly based on the average weight for each of the nine cohorts of the first example. A single dilution of BIO 300 oral suspension at a concentration of 65 mg/mL was prepared each week and used for that week's dosing, altering the dose by adjusting the volume of drug delivered. Animal weights and tumor volumes were measured twice weekly. A radiation dose of 12.5 Gy was given when the average tumor volume of the respective cohort reached ˜450 mm³. It was anticipated that this radiation dose should generate sufficient tumor growth delay while not curing the animals of their tumors. This is essential to determine if BIO 300 acts as a radiosensitizing agent in conjunction with radiation on tumor growth.

The animals were dosed for 10 weeks (5 d/wk) TABLE ONE. At the ten week point a number of animals in the study were euthanized due to large tumors that had become necrotic. Cohorts treated with vehicle only also had necrotic tumors, indicating that tumor necrosis was unrelated to BIO 300 treatment. The data presented here includes all data through the ten-week time point prior to the loss of animals from IACUC-mandated euthanization. All tumor volume data and animal weight data were analyzed through the ten weeks of dosing. Animals selected for histopathological analysis were euthanized at approximately the same time relative to the timing of the radiation dose.

Following euthanasia organ weights were collected for the lungs, uterus, and GI tract. In addition, the tumor was resected and weighed. We also collected a section of the skin near the tumor site which was subjected to histopathological analysis to evaluate the BIO 300 protective effects on normal skin tissue.

Tumor Volumes:

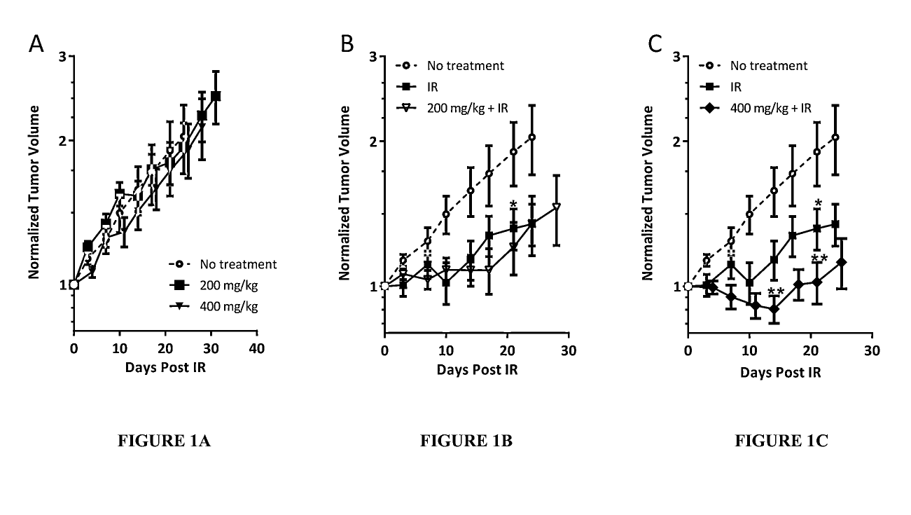

Following ten weeks of BIO 300 dosing (5 d/wk) the analysis of tumor volumes calculated using the tumor dimensions collected twice weekly was completed. When the average tumor volume of the group to be treated reached 400-425 mm³, all of the animals in that group received a single 12.5 Gy dose of radiation from a 137Cs irradiator. The 2 cm wide radiation path used in this example not only irradiated the tumor but also irradiated the lungs and possibly the upper GI tract. The example was designed in this way to allow a histological assessment of irradiated normal tissue enabling the evaluation of the protective effects of BIO 300 on normal tissue. The results of the analysis of calculated tumor volumes are shown in FIGS. 1A, 1B and 1C. The tumor volumes (mean±SEM) were normalized and plotted versus the days post tumor irradiation. TABLE TWO infra includes all mean tumor volumes for each group for the length of the example. * in TABLE TWO indicates the day of tumor irradiation for the group. The Because the cohorts were irradiated on different days, and the day of irradiation was different relative to the days volumes were measured, day 0 on the plot is within 48 hours after tumor irradiation.

TABLE TWO

| | | | BIO 300 | BIO 300 | BIO 300 | BIO 300 |
|---|---|---|---|---|---|---|
| | Vehicle | Vehicle IR | (200 mg/kg) IRr | (400 mg/kg) IRr | (200 mg/kg) | (400 mg/kg) |
| | Group 2 (n = 12) | Group 3 (n = 10) | Group 4 (n = 10) | Group 5 (n = 12) | Group 6 (n = 12) | Group 7 (n = 12) |
| Day | Mean ± SEM (mm³) | | | | | |
| 0 | 71.6 ± 9.9 | 48.4 ± 5.4 | 31.1 ± 4.6 | 23.1 ± 3.9 | 27.8 ± 6.7 | 21.5 ± 6.5 |
| 3 | 58.2 ± 5.3 | 57.6 ± 8.4 | 49.3 ± 4.1 | 49.3 ± 3.3 | 58.3 ± 4.7 | 64.4 ± 15.0 |
| 7 | 74.7 ± 8.8 | 78.5 ± 11.3 | 76.4 ± 7.8 | 76.8 ± 5.9 | 87.7 ± 7.9 | 94.6 ± 15.5 |
| 10 | 91.3 ± 14.3 | 83.0 ± 6.4 | 116.2 ± 14.1 | 85.4 ± 7.2 | 96.7 ± 6.9 | 102.3 ± 16.7 |
| 14 | 110.3 ± 15.7 | 107.1 ± 9.4 | 142.9 ± 16.2 | 102.9 ± 7.5 | 128.4 ± 9.2 | 139.2 ± 23.3 |
| 17 | 124.3 ± 21.2 | 123.5 ± 12.2 | 181.3 ± 25.7 | 128.4 ± 12.1 | 154.3 ± 13.8 | 174.8 ± 27.6 |
| 21 | 162.2 ± 27.8 | 154.6 ± 20.7 | 216.7 ± 31.6 | 179.1 ± 20.4 | 188.6 ± 18.0 | 193.9 ± 38.4 |
| 24 | 175.9 ± 30.3 | 185.9 ± 25.7 | 255.2 ± 37.5 | 223.7 ± 30.1 | 247.3 ± 26.0 | 233.6 ± 54.8 |
| 28 | 205.1 ± 32.9 | 241.5 ± 41.1 | 342.7 ± 51.4 | 288.5 ± 49.8 | 302.8 ± 32.4 | 277.5 ± 65.3 |
| 31 | 223.4 ± 38.3 | 257.9 ± 43.2 | 418.7 ± 73.9 | 328.0 ± 57.7 | 357.9 ± 43.9 | 309.4 ± 67.6 |
| 35 | 200.2 ± 34.6 | 278.6 ± 58.0 | *427.8 ± 77.4* | 361.0 ± 66.9 | *418.3 ± 61.2* | 344.2 ± 71.2 |
| 38 | 277.9 ± 55.3 | 367.6 ± 70.8 | 460.2 ± 88.9 | *440.0 ± 91.2* | 511.8 ± 83.8 | *399.4 ± 77.2* |
| 42 | *305.3 ± 64.5* | *391.9 ± 78.5* | 445.4 ± 88.7 | 455.6 ± 102.4 | 582.4 ± 103.5 | 437.1 ± 85.4 |
| 45 | 346.4 ± 75.9 | 404.8 ± 86.9 | 464.9 ± 91.7 | 457.7 ± 110.9 | 669.3 ± 120.3 | 525.6 ± 102.9 |
| 49 | 381.1 ± 88.8 | 454.9 ± 103.8 | 470.1 ± 96.3 | 437.7 ± 108.4 | 668.6 ± 128.5 | 564.6 ± 117.1 |
| 52 | 434.4 ± 106.3 | 437.5 ± 114.7 | 477.3 ± 103.9 | 445.3 ± 117.0 | 766.6 ± 150.7 | 640.9 ± 136.3 |
| 56 | 472.4 ± 120.7 | 481.5 ± 123.7 | 521.5 ± 113.0 | 503.7 ± 131.2 | 825.1 ± 184.6 | 709.3 ± 154.8 |
| 59 | 520.3 ± 144.2 | 539.1 ± 136.4 | 585.9 ± 133.1 | 535.6 ± 151.1 | 886.4 ± 204 | 767.4 ± 172.8 |
| 63 | 585.4 ± 168.9 | 568.2 ± 151.2 | 618.9 ± 143.8 | 608.8 ± 180.2 | 1033.8 ± 232.9 | 849.7 ± 196.6 |
| 66 | 643.5 ± 198.0 | 575.6 ± 151.3 | 659.4 ± 163.2 | 686.9 ± 206.4 | 1122.8 ± 249.2 | 939.5 ± 222.5 |

As shown in FIG. 1A, BIO 300 (200 mg/kg or 400 mg/kg) as a monotherapy had no effect on tumor volume. A one-way ANOVA followed by Dunnett's post-hoc multiple comparisons test was employed to look for statistical significant differences. The statistical test was performed only on those days in which recorded volumes for all groups we′re available (e.g., day 7, 14, and 21 in FIG. 1C). As shown in FIGS. 1B and 1C tumor radiation alone and tumor radiation with BIO 300 treatment had a profound effect on tumor volume. These data were also analyzed using a linear mixed model method comparing the rate of tumor growth between the different study groups. Compared to vehicle alone, BIO 300 in combination with radiation treatment had a slower rate of tumor growth (p=0.046).

Tumor Growth Delay:

Tumor growth delay (TGD) was determined for all treatment groups and compared to the vehicle group (no treatment). TGD was determined and analyzed in accordance with the methods described in Zhang, X., et al., In Vitro and In Vivo Study of a Nanoliposomal Cisplatin as a Radiosensitizer. Int J Nanomedicine, 2011. 6: p. 437-44. For the current analysis TGD was defined as the difference between T(1.25V) of treated tumors compared to untreated tumors. T(1.25V) is the number of days needed for tumor growth from the original size on the day of radiation to a volume that was 25% larger. The results of that analysis are shown in the TABLE THREE below.

TABLE THREE

| | T(1.25 V) (days) | TGD (days) | % TGD increase |
|---|---|---|---|
| Group | [Min-Max] | [Min-Max] | [Min-Max] |
| Vehicle | 4.36-9.55 | 0 | ~ |
| Vehicle + IR | 14.21-22.63 | 9.86-13.09 | ~ |
| 200 mg/kg + IR | 18.19-29.65 | 13.84-20.10 | 40-54% |
| 400 mg/kg + IR | 24.07-36.88 | 19.71-27.34 | 100-109% |

The data from the tumor volume growth characteristics and the calculations of TGD indicate that BIO 300 does not have a therapeutic effect on tumor growth by itself but does further sensitize the tumor to radiotherapy. BIO 300 (400 mg/kg) in conjunction with tumor irradiation, increased tumor growth delay compared to radiation treatment alone (FIG. 1C) by 100-109%. The tumor growth curves for BIO 300 (200 mg/kg) suggest an increase in tumor growth delay, which is supported by the 40-54% increase found in TGD. The SEM (+/−) of the tumor volume was used to estimate the range in TGD.

Together these data suggest that BIO 300 further sensitizes the tumor to radiation, increasing the efficacy of the radiotherapy.

Animal Weights:

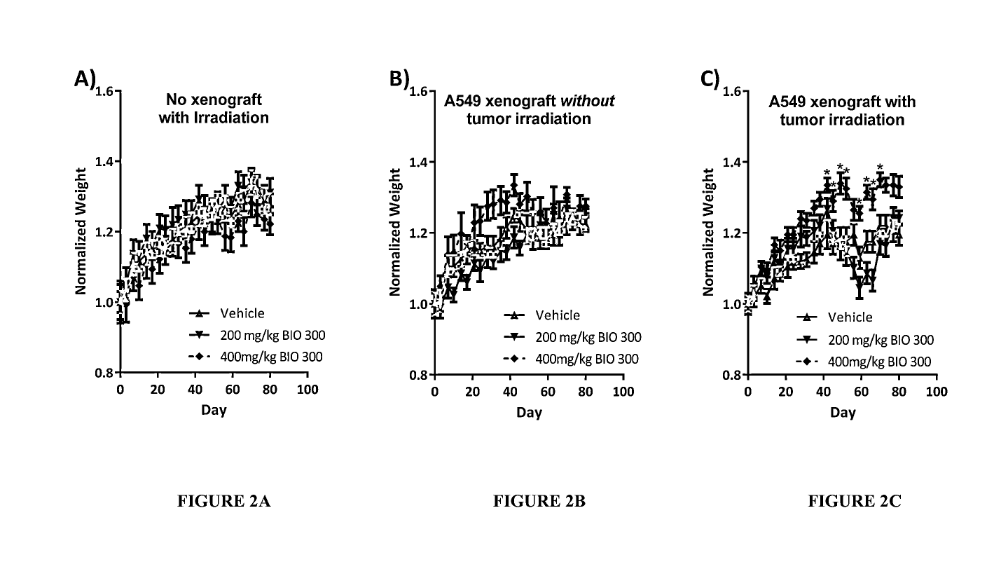

The weights of the mice were collected twice weekly. Referring to FIGS. 2A, 2B, and 2C, the animal weights were normalized and plotted versus days on study. The data were divided into subsets and analyzed based on whether the group had an A549 xenograft and whether the group received radiation treatment. The data were plotted as the mean±SEM and to look for statistical differences a one-way ANOVA with a Dunnett's multiple comparisons post-hoc analysis was completed. Each of the cohorts that received irradiation were exposed to a single 12.5 Gy dose at day 40±2 days. The three groups that did not receive an A549 xenograft, but were irradiated, had no significant differences in animal body weight over the ten weeks (FIG. 2A). Referring to FIG. 2A, a flattening of the curve and a transient decrease in animal weight can be observed for all three groups following radiation treatment. The results of the cohorts with A549 xenografts and (with or without) tumor irradiation are noticeably different. In the animals that did not receive irradiation (FIG. 2B) the cohort receiving 400 mg/kg BIO 300 had an apparent increase in weight gain between days 20 and 40 compared to the vehicle and 200 mg/kg groups. This analysis was done by comparing the mean body weights on individual days. When this data was analyzed using a linear mixed model method to compare the trajectory of weight gain overtime, BIO 300 dosed at 400 mg/kg had a greater rate of weight increase (p=0.0003). We conclude that the higher BIO 300 dose (400 mg/kg), in the presence of an A549 tumor, results in increased weight over the course of the study. Finally, in the animals that had an A549 xenograft and received tumor irradiation, the cohort receiving 400 mg/kg BIO 300 were better able to tolerate the radiation treatment (FIG. 2C). These animals were able to better sustain their weight following radiation treatment. In the vehicle and 200 mg/kg BIO 300 cohorts, the inflection point in the curve is quite obvious while the weight in the 400 mg/kg BIO 300 cohort was maintained following tumor irradiation. A statistical analysis of the data in FIG. 2C found that the 400 mg/kg cohort was different than vehicle (and 200 mg/kg BIO 300) at the points marked on the plot (* p<0.05). This finding was confirmed using a linear mixed model analysis. BIO 300 dosed at 400 mg/kg had a faster weight gain trajectory over time compared to mice treated with BIO 300 vehicle (p=0.051). This maintenance in body weight within the 400 mg/kg BIO 300 cohort that received tumor irradiation cannot be explained by tumor weight as those animals had the slowest growing tumors following radiation (FIG. 1C).

Wet Lung Weights:

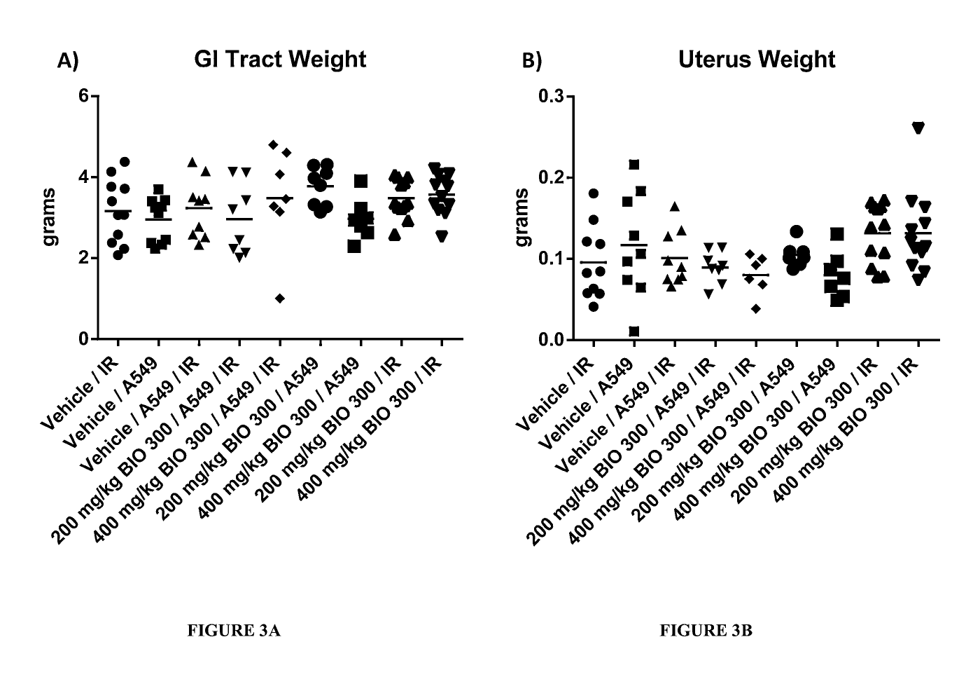

In experimental animal models the wet-lung weight is used to assess the level of edema/congestion in the lung. This makes the weight lung weight a good measure of radiation-induced pneumonitis and fibrosis in the lung tissue. The wet weight of the lungs were determined at the time of euthanasia. The wet lung weights were collected 6-8 weeks post radiation. The irradiated animals that received BIO 300 vehicle did have a larger mean wet lung weight (FIG. 3A). However, this observation is confounded in that the same trend is not observed in the irradiated animals that received the A549 xenograft and BIO 300 vehicle. When comparing the groups that received thoracic irradiation, but no A549 xenograft, a trend was observed suggesting that BIO 300 dosing can mitigate the radiation-induced increase in wet lung weight (FIG. 3B). These data suggest that BIO 300 can mitigate the induction of radiation-induced pneumonitis.

Post Mortem Tissue Weights:

To further assess the effects of 10 weeks of daily BIO 300 dosing (5 d/wk) the wet tissue weights of the uterus and GI tract were collected following euthanasia (FIGS. 3A and 3B). The upper GI tract is within the path of radiation making this an ideal tissue to further evaluate the protective effects of BIO 300 on normal tissue and also to monitor for detrimental effects form continued BIO 300 administration (safety). To assess gross pathology and morphology the GI tract (esophagus/stomach/intestines) was weighed at the time of euthanasia (FIG. 3A). There was a wide spread in the weights, but no obvious differences between the experimental cohorts. To rule out estrogenic effects of prolonged BIO 300 oral administration over the ten week study the uteri in the study mice were removed and weighed at the time of euthanasia. As shown in FIG. 3B, we did not detect significant differences between the experimental cohorts. While the recorded weights covered a wide range within each cohort, we can conclude that in this study the phytoestrogen genistein did not dramatically affect the wet uterine weights of the experimental animals.

Immunohistochemistry:

To evaluate tumor cell proliferation immunohistochemical staining for the nuclear protein Ki67 was completed on tumor sections from one mouse from each of the groups that had A549 tumors in the first experiment. Ki67 is a protein associated with cellular proliferation and is widely used as a cell proliferation biomarker. Following euthanasia tumor sections from each group were stained with a Ki67 antibody and the Ki67 positive foci visualized and quantified by fluorescent microscopy in accordance with the procedure described in Jenrow, K. A., et al., Combined Atorvastatin and Ramipril Mitigate Radiation-Induced Impairment of Dentate Gyrus Neurogenesis. J Neurooncol, 2011. 101(3): p. 449-56. For this study the tumors were harvested approximately 6 weeks post-irradiation during regrowth. The tumors within the different groups all regrew at approximately the same rate. The distribution of cells expressing Ki67 (red) and the intensity of staining within the cells were similar among the different treatment groups. All of the cell nuclei were labeled with DAPI (blue).

Histopathology (Skin, Tumor, Lung, Uterus, and Esophagus):

Additional tissues from this study were analyzed histologically to further investigate the protective effects of BIO 300 on normal tissues and also determine if continued daily administration of BIO 300 resulted in any adverse effects on the tissues analyzed. The examined tissues included the lungs, GI tract (esophagus), uterus, tumor, and the skin near the A549 tumor xenograft. In this example the tumor was placed such that the lungs would be in the radiation path, allowing an evaluation of the radioprotective effects of BIO 300 in the normal lung. All tissues were analyzed microscopically following H&E staining. For these analyses 5 tumors, 5 lung samples, and 5 skin samples were analyzed from each experimental group. In addition, three uterus samples and three esophagus samples from each experimental group were analyzed. All tissue samples were prepared, sectioned, and stained with H&E. Tissues were analyzed for gross morphology and H&E sections were evaluated for inflammatory cells, capillary distension, and congestion. A veterinary pathologist scored the severity and distribution of anomalies observed. Severity was scored on a scale from 0-4; (0=unremarkable/normal, 1=minimal, 2=mild, 3=moderate, 4=severe), the distribution was scored as being focal (F), multifocal (M), diffuse (D), or present (P). The conclusions drawn from the histopathological analyses of the different tissues are shown in TABLE FOUR below.

TABLE FOUR

| TISSUE | HISTOPATHOLOGY CONCLUSIONS |
|---|---|
| LUNG | Except for Group I mice, increased alveolar septal wall thickness with |
| | increased collagen was observed in mice treated with treatment regimens |
| | that included radiation. In Group 3 treated with vehicle control, tumor |
| | implant, with radiation, mild to moderate increased septal thickness was |
| | observed in 3/5 mice whereas in group 5 (BIO 300 @ 400 mg/kg, tumor |
| | implant, with radiation) and group 9 (BIO 300 @ 400 mg/kg, no tumor, |
| | with radiation), mild to moderate increased septal thickness was observed |
| | in 1/5 and 0/5 mice, respectively. Lung damage as assessed by septal |
| | thickness was less prevalent in mice receiving the highest dose of BI0 |
| | 300, i.e. 400 mg/kg, compared with that in mice receiving vehicle. Other |
| | findings included increased alveolar macrophages, alveolar edema or |
| | fibrin, and inflammatory cell infiltrates were low in incidence and |
| | considered to be unrelated to any particular treatment regimen. |
| SKIN | Microscopic observations included minimal mixed cellular or neutrophilic |
| ADJACENT TO | inflammation and decreased or increased epidermal thickness. These |
| THE TUMOR | findings were scattered among the various treatment combinations of |
| | vehicle control, BI0 300, tumor implant, and radiation and were |
| | considered to have no relationship to any particular treatment regimen. |
| UTERUS & | The presence of bacteria in the esophageal lumen was observed in all |
| ESOPHAGUS | groups and was considered to be an incidental finding. However, |
| | decreased mucosa) cornification observed in one Group 8 mouse and |
| | decreased epithelial thickness observed in one Group 9 mouse, which |
| | were treated with BI0 300 @200 mg/kg with radiation and BI0 300 @400 |
| | mg/kg with radiation, respectively, had an uncertain relationship to |
| | treatment. |
| TUMOR | Cystic areas of degeneration and necrosis with or without inflammation |
| | were observed in tumor implants in all animals in all six groups and was |
| | considered to be unrelated to any particular treatment regimen. |

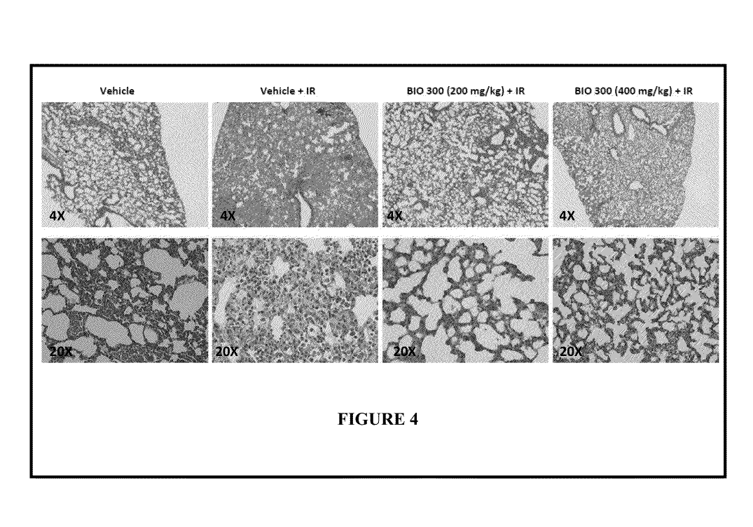

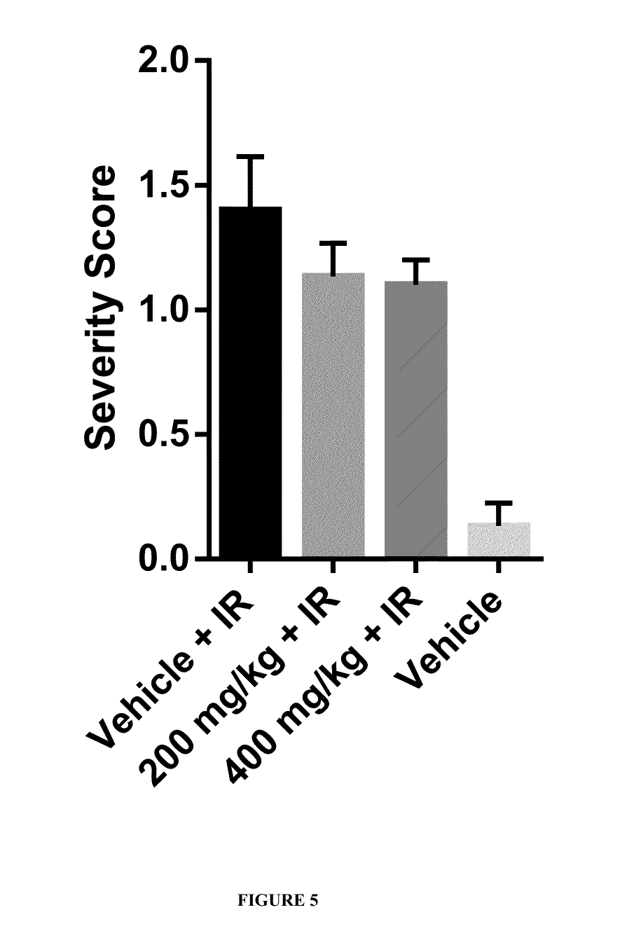

Histopathology analysis of skin, uterus, and esophagus tissue sections did not find any adverse tissue response that was correlated with BIO 300 administration or dose of drug. The few pathological findings that were noted did not correlate with BIO 300 administration. These results are consistent with the organ weights for the uterus and GI tract. Together, these data support the safety of extended daily BIO 300 oral dosing. Consistent with the results of the Ki67 immunohistochemical staining, and the fact that tumor sections were analyzed during the regrowth phases, no differences in tumor histopathology were observed. In contrast, in the lungs of the animals with tumors that were treated with radiation, but did not receive BIO 300 numerous adverse histopathological findings were identified. These included infiltration of macrophages, congestion and edema, increased septal wall thickness, alveolar fibrin, and pneumocyte hypertrophy (FIG. 4). All of these findings are consistent with radiation-induced pulmonary damage. Importantly, continued BIO 300 administration had no adverse effect on the lungs of the animals that did not receive radiation, indicating that BIO 300 oral administration is safe. In contrast, the animals that received vehicle alone or BIO 300 in addition to radiation were found to have pulmonary pathology that was nearly unremarkable. Referring to FIG. 4, microscopic analysis of the pulmonary tissue from the animals that received radiation and vehicle only had clear signs of congestion, infiltration of inflammatory cells, and thickening of the septal wall. These histopathological findings were not evident in the vehicle group that did not receive radiation treatment or in the groups that received radiation but were treated with BIO 300. BIO 300 mitigates the formation of radiation-induced pulmonary damage in normal lung tissue. From the pulmonary pathology, clearly the animals that were treated with BIO 300 and radiation closely resembled that pathology of animals treated with vehicle alone without radiation. Radiation had fewer effects on the lungs of animals treated with BIO 300. Referring to FIG. 5, in order to further evaluate the radioprotective effects of BIO 300 on the lung the severity scores from the histopathological analyses were analyzed and compared among the experimental groups. An average severity score was determined for each animal. To ensure a fair comparison the severity score from each animal was equally weighted. As seen in FIG. 5, this analysis supports the conclusion that BIO 300 has radioprotective effects on irradiated lung tissue.

Findings:

Daily oral dosing of BIO 300 prior to and post a single dose of tumor radiation mitigates the formation of radiation-induced pulmonary damage. Continued daily administration of BIO 300 at doses up to 400 mg/kg in mice does not protect tumor tissue from radiation-induced killing. When administered daily for six weeks prior to tumor irradiation and for four weeks after radiation, BIO 300 further sensitizes NSCLC tumors in this xenograft model to radiation-induced killing. BIO 300 dosed at 400 mg/kg had a more profound effect on tumor radiosensitization in this study. Of note, determination of tumor growth delay also suggested that BIO 300 dosed at 200 mg/kg was able to sensitize the tumor to radiation. BIO 300 dosed at 400 mg/kg supported the maintenance of a stable body weight following radiation treatment. Without intending to be limited thereby, it is believed that this effect is mediated by a more rapid rate of weight gain prior to radiation in the BIO 300 groups. Histopathological analysis from animals dosed daily (5 d/wk) with BIO 300 for up to 10 weeks found no adverse effects of BIO 300 on the tissues analyzed. Tissues that were analyzed include the GI tract (esophagus and terminal ileum), skin, uterus, and the lungs of non-irradiated animals. These findings support the safety of extended daily BIO 300 oral dosing.

### Example 2

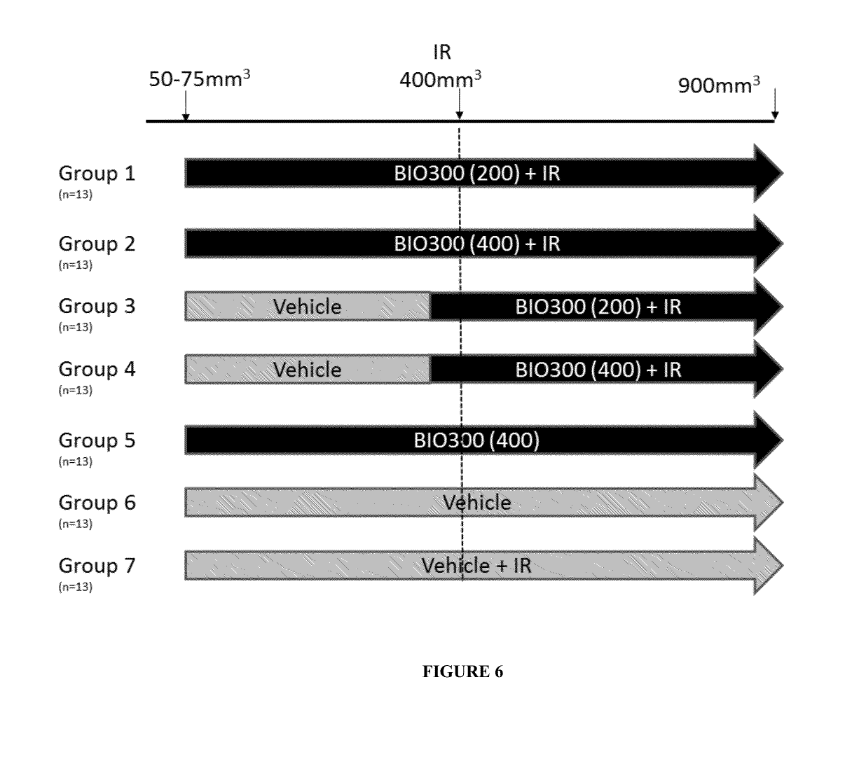

The design, process and procedures employed in Example 2 followed those employed in Example 1 unless otherwise noted. The experimental design for Example 2 is shown in FIG. 6.

The A549 cells were implanted and tumors allowed to become established prior to the start of BIO 300 dosing. Tumors were placed in the subcutaneous space in the rear flank. Following tumor initiation the tumors were randomized into cohorts such that each cohort had a similar mean tumor volume and standard deviation. At the time of randomization the average tumor volume for each experimental cohort was approximately 75 mm³. To determine if there is a correlation between tumor size at the start of treatment and BIO 300 efficacy we initiated BIO 300 treatment 7 days prior to tumor irradiation for two of the groups (3 and 4). In the remaining groups BIO 300 was dosed for 31 consecutive days prior to tumor irradiation.

A549 Xenograft Model and Tumor Location:

2×10⁶ A549 cells were implanted in a 1:1 mixture with Matrigel (100 ul total), seeded within the subcutaneous space in the rear flank on the dorsal side of the mouse. The 2 cm wide radiation path used in this example will not only irradiate the tumor but may also irradiate the uterus and parts of the GI tract. This will allow an assessment of BIO 300 efficacy on the tumor itself, in combination with radiation, and also the radioprotective effects on these surrounding normal tissues. A549 NSCLC cells were implanted into 91 CD1 female athymic nude mice. Of the 91 implanted mice, 77 (85%) developed tumors. This example included a total of 91 mice divided into 7 groups (13 mice/cohort). The groups are shown in FIG. 6. The mice were dosed 7 days/week.

### In-Life Study Activities:

Tumor Volumes:

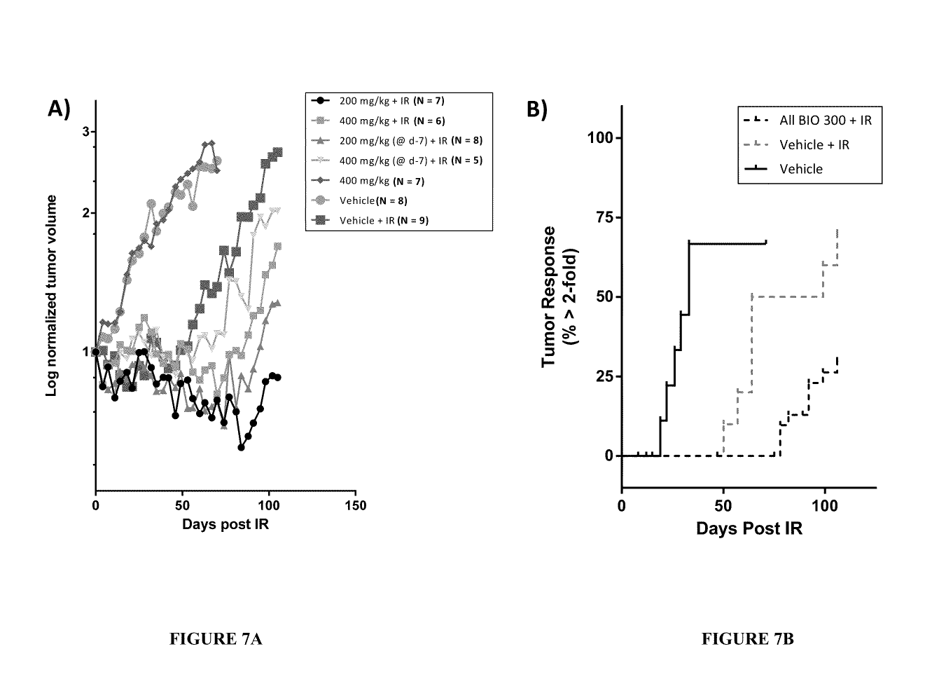

BIO 300 dosing (7 d/wk) continued for nineteen consecutive weeks. During that time tumor volumes were calculated using the tumor dimensions collected twice weekly. When the average tumor volume of the group to be treated reached approximately 400 mm3 all of the animals in that group received a single 12.5 Gy dose of radiation from a 137Cs irradiator. All animals that were to receive tumor irradiation were dosed with the single 12.5 Gy dose on the same day. The length of this study resulted in loss of animals due to necrotic tumors form all experimental groups. There was no relationship between treatment group and veterinary mandates to euthanize animals. Moreover, the long duration of the study resulted in increased variability of calculated tumor volumes the farther removed from the day of tumor irradiation. For these reasons only the mean tumor volumes for those animals that completed the study are depicted in FIG. 7A. TABLE FIVE below includes the mean tumor volume and standard error of the mean for all groups and all days. * in TABLE FIVE indicates the day of tumor irradiation for the group.

TABLE FIVE

| | | | BIO 300 | BIO 300 | | | |
|---|---|---|---|---|---|---|---|
| | BIO 300 | BIO 300 | (200 mg/kg) | (400 mg/kg) | BIO 300 | | |
| | (200 mg/kg) IR | (400 mg/kg) IR | @ day-7 IR | @ day-7 IR | (400 mg/kg) | Vehicle | Vehicle IR |
| | Group 1 (n = 7) | Group 2 (n = 6) | Group 3 (n = 8) | Group 4 (n = 5) | Group 5 (n = 7) | Group 6 (n = 8) | Group 7 (n = 9) |
| Day | Mean ± SEM (mm³) | | | | | | |
| 0 | 73.3 ± 5.1 | 73.6 ± 4.5 | 60.8 ± 12.6 | 69.9 ± 11.1 | 67.1 ± 8.9 | 87.3 ± 4.8 | 77.5 ± 7.3 |
| 3 | 83.6 ± 9.9 | 98.9 ± 3.4 | 98.8 ± 14.6 | 93.1 ± 15.4 | 110.9 ± 29.3 | 131.6 ± 12.6 | 95.8 ± 8.2 |
| 7 | 95.7 ± 6.8 | 99.0 ± 11.4 | 117.3 ± 26.4 | 135.8 ± 18.6 | 134.9 ± 41.5 | 156.4 ± 18.7 | 118.2 ± 11.6 |
| 10 | 116.3 ± 13.7 | 125.5 ± 9.0 | 126.5 ± 27.0 | 167.3 ± 21.8 | 143.4 ± 40.4 | 173.1 ± 18.4 | 143.1 ± 13.8 |
| 14 | 153.9 ± 31.9 | 163.1 ± 17.1 | 189.0 ± 39.2 | 224.1 ± 38.5 | 161.3 ± 42.2 | 186.1 ± 26.6 | 160.8 ± 18.1 |
| 17 | 186.0 ± 40 | 193.2 ± 23.0 | 222.1 ± 42.3 | 258.5 ± 64.6 | 225.0 ± 38.7 | 217.8 ± 28.9 | 201.8 ± 24.4 |
| 21 | 257.0 ± 44.2 | 198.1 ± 25.7 | 249.8 ± 57.9 | 322.6 ± 84.8 | 246.5 ± 58.3 | 278.4 ± 43.7 | 264.0 ± 52.7 |
| 24 | 239.0 ± 45.8 | 225.3 ± 30.0 | 253.0 ± 56.6 | 345.6 ± 90.3 | 267.3 ± 63.9 | 302.8 ± 42.8 | 292.1 ± 55.1 |
| 28 | 354.9 ± 66.2 | 337.9 ± 51.6 | 326.5 ± 75.8 | 465.0 ± 120.2 | 362.9 ± 97.7 | 390.8 ± 58.1 | 318.3 ± 67.9 |
| 31 | *376.6 ± 86.1* | *385.6 ± 68.3* | *365.7 ± 91.6* | *505.0± | *417.6± | *418.5 ± 52.2* | *357.6 ± 70.9* |
| 35 | 316.8 ± 74.9 | 327.2 ± 57.3 | 312.0 ± 87.5 | 429.8 ± 119.1 | 484.3 ± 124.5 | 451.1 ± 62.6 | 360.1 ± 70.2 |
| 38 | 349.2 ± 79.4 | 360.2 ± 60.4 | 303.2 ± 78.5 | 417.7 ± 107.3 | 480.1 ± 144.5 | 447.3 ± 66.7 | 335.7 ± 67.8 |
| 42 | 299.6 ± 77.1 | 364.7 ± 69.4 | 312.4 ± 88.1 | 406.7 ± 126.1 | 482.4 ± 126.3 | 469.7 ± 72.3 | 350.3 ± 70.0 |
| 45 | 325.3 ± 74.7 | 399.8 ± 77.6 | 331.4 ± 93.5 | 502.1 ± 167.3 | 509.8 ± 140.1 | 511.6 ± 70.4 | 319.0 ± 69.4 |
| 49 | 340.2 ± 77.9 | 388.8 ± 78.9 | 320.9 ± 92.9 | 488.5 ± 165.4 | 614.5 ± 161.4 | 599.2 ± 89.5 | 300.3 ± 53.8 |
| 52 | 314.2 ± 76.6 | 387.0 ± 84.2 | 343.8 ± 91.9 | 538.8 ± 177.2 | 683.4 ± 163.7 | 659.7 ± 101.6 | 300.8 ± 68.0 |
| 56 | 375.4 ± 87.5 | 436.2 ± 95.8 | 348.5 ± 96.7 | 554.0 ± 204.5 | 701.6 ± 194.0 | 683.7 ± 100.6 | 333.5 ± 73.7 |
| 59 | 377.0 ± 94.0 | 457.6 ± 91.2 | 337.6 ± 91.5 | 530.4 ± 190.4 | 726.9 ± 189.2 | 741.3 ± 121.6 | 318.0 ± 67.3 |
| 63 | 348.4 ± 78.2 | 425.8 ± 91.0 | 325.5 ± 92.6 | 495.4 ± 180.2 | 707.0 ± 191.0 | 876.6 ± 148.0 | 382.6 ± 90.1 |
| 66 | 321.0 ± 66.1 | 382.1 ± 80.4 | 300.7 ± 86.8 | 562.3 ± 233.7 | 790.9 ± 244.6 | 764.7 ± 141.9 | 375.0 ± 100.2 |
| 70 | 332.0 ± 77.1 | 364.6 ± 71.0 | 301.9 ± 78.7 | 492.3 ± 157.6 | 806.7 ± 256.5 | 835.0 ± 149.7 | 348.8 ± 78.4 |
| 73 | 331.1 ± 75.0 | 380.7 ± 69.6 | 322.4 ± 94.6 | 490.8 ± 139.4 | 847.1 ± 294.9 | 862.1 ± 170.3 | 329.5 ± 72.1 |
| 77 | 274.2 ± 61.6 | 359.7 ± 79.7 | 306.3 ± 80.1 | 450.6 ± 130.7 | 952.0 ± 324.6 | 928.0 ± 183.2 | 334.3 ± 74.0 |
| 80 | 321.8 ± 77.7 | 401.7 ± 90.9 | 329.0 ± 91.6 | 524.0 ± 154.0 | 987.8 ± 345.7 | 916.2 ± 159.7 | 360.9 ± 82.6 |
| 84 | 327.5 ± 77.1 | 390.9 ± 80.5 | 276.1 ± 76.4 | 501.8 ± 155.8 | 1017.6 ± 377.3 | 965.1 ± 190.9 | 368.0 ± 85.4 |
| 87 | 298.5 ± 71.6 | 348.7 ± 88.2 | 276.8 ± 80.0 | 484.6 ± 147.2 | 1041.1 ± 413.7 | 865.9 ± 170.7 | 410.0 ± 116.6 |
| 91 | 277.0 ± 65.1 | 335.3 ± 78.5 | 304.4 ± 99.6 | 540.0 ± 160.9 | 1076.4 ± 421.2 | 1054.9 ± 229.1 | 443.6 ± 128.2 |
| 94 | 292.2 ± 65.8 | 352.5 ± 87.3 | 272.8 ± 86.2 | 547.3 ± 143.4 | 1174.7 ± 484.3 | 1052.6 ± 236.8 | 499.5 ± 137.9 |
| 98 | 271.2 ± 58.6 | 360.0 ± 93.2 | 278.7 ± 91.2 | 512.8 ± 137.5 | 1182.6 ± 503.2 | 1044.8 ± 275.9 | 479.2 ± 119.7 |
| 101 | 296.2 ± 66.7 | 312.7 ± 90.2 | 285.7 ± 91.9 | 554.0 ± 155.2 | 1032.2 ± 429.3 | 1086.5 ± 289.3 | 494.7 ± 134.0 |
| 105 | 264.8 ± 66.6 | 339.0 ± 105.2 | 253.1 ± 86.4 | 549.9 ± 159.4 | | | 593.5 ± 162.3 |
| 108 | 300.6 ± 70.2 | 380.7 ± 115.4 | 362.0 ± 114.1 | 723.1 ± 195.0 | | | 530.3 ± 135.0 |
| 112 | 279.3 ± 77.6 | 388.5 ± 134.5 | 277.4 ± 85.4 | 717.6 ± 203.7 | | | 589.3 ± 164.9 |
| 115 | 233.8 ± 64.4 | 379.0 ± 127.6 | 325.3 ± 122.8 | 665.9 ± 180.5 | | | 701.9 ± 197.6 |
| 119 | 247.4 ± 60.5 | 419.9 ± 153.1 | 304.5 ± 114.9 | 625.1 ± 179.9 | | | 700.8 ± 186.2 |
| 122 | 264.2 ± 65.0 | 462.4 ± 164.8 | 336.2 ± 124.9 | 903.4 ± 243.8 | | | 743.5 ± 201.2 |
| 126 | 283.7 ± 66.4 | 474.3 ± 166.2 | 376.4 ± 141.3 | 989.1 ± 274.8 | | | 771.0 ± 206.4 |
| 129 | 324.6 ± 80.5 | 566.9 ± 195.3 | 427.5 ± 157.3 | 943.3 ± 256.3 | | | 914.1 ± 245.7 |
| 133 | 334.5 ± 95.6 | 594.7 ± 205.2 | 464.5 ± 175.6 | 1020.5 ± 293.7 | | | 945.6 ± 255.0 |
| 136 | 331.7 ± 105.5 | 653.4 ± 251.0 | 468.1 ± 177.3 | 1019.3 ± 294.5 | | | 968.4 ± 242.6 |

The variability in tumor size increased with increasing time in the study. All tumor volume data, regardless of length of time on study, was used in the statistical analyses described below. Tumor volume was calculated twice weekly for a duration of 105 days post radiation treatment. The plot of mean tumor volumes clearly indicated that the addition of BIO 300 to radiation treatment results in an improved tumor growth inhibition and increased TGD (FIG. 7A). BIO 300 in combination with radiation therapy does not protect the tumor. The data suggests BIO 300 dosed at 200 mg/kg is more efficacious than the higher dose and initiation of dosing early (smaller tumors) may provide a modest improvement in response. A Cox regression analysis was performed on the data to determine the beneficial effect of combining BIO 300 and radiation therapy on tumor growth (FIG. 7B). In this initial analysis all groups that received BIO 300 and tumor irradiation were combined. For this statistical analysis the event was defined as a doubling in tumor size, using the tumor volume on the day of radiation as the baseline. Combining all groups, the addition of BIO 300 to radiation treatment reduced the chance of the tumor from doubling (p=0.008). The risk of the tumor doubling was reduced by 74% when BIO 300 was added to radiation (HR=0.26, 95% CI: 0.10-0.95).

Tumor Growth Delay:

Tumor growth delay (TGD) was determined for all treatment groups and compared to the vehicle group (no treatment). TGD was determined and analyzed in accordance with the methods described in Zhang, X., et al., In Vitro and In Vivo Study of a Nanoliposomal Cisplatin as a Radiosensitizer. Int J Nanomedicine, 2011. 6: p. 437-44. For the current analysis TGD was defined as the difference between T(1.5V) of treated tumors compared to untreated tumors. T(1.5V) is the number of days needed for tumor growth from the original size on the day of radiation to a volume that was 50% larger. The results of that analysis are shown in the TABLE SIX below.

TABLE SIX

| | T(1.5 V) (days) | TGD (days) | % TGD increase |
|---|---|---|---|
| Group | [Min-Max] | [Min-Max] | [Min-Max] |
| Vehicle | 15.33-28.07 | 0 | ~ |
| Vehicle + IR | 60.79-83.33 | 45.46-55.26 | ~ |
| 200 mg/kg + IR | 124.96-160.3 | 109.63-132.23 | 139-141% |
| 200 mg/kg (@ | 104.43-131.03 | 89.09-102.96 | 86-96% |
| d-7) + IR | | | |
| 400 mg/kg + IR | 86.95-121.86 | 71.62-93.79 | 58-70% |
| 400 mg/kg (@ | 74.92-105.82 | 59.59-77.75 | 31-41% |
| d-7) + IR | | | |

The addition of BIO 300 to radiotherapy dramatically increases the TGD. Both BIO 300, dosed at either 200 or 400 mg/kg, in conjunction with tumor irradiation, increased tumor growth delay compared to radiation treatment alone (FIG. 7B). Compared to radiation alone, the addition of BIO 300 increased TGD from 31-141%. The SEM (+/−) of the tumor volume was used to estimate the range in TGD. BIO 300 further sensitizes the tumor to radiation, increasing the effectiveness of the radiotherapy.

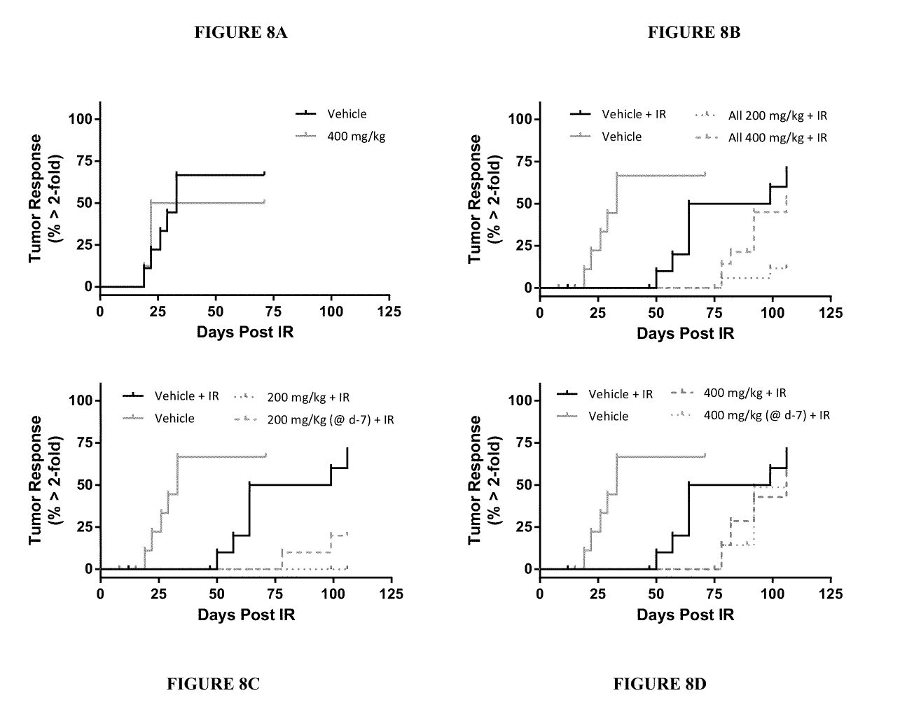

A detailed statistical analysis of the tumor growth data and associated tumor growth delay was completed to further evaluate the significance of the data. Cox regression analysis was used for statistical comparisons. For this statistical analysis the event was defined as a doubling in tumor size, using the tumor volume on the day of radiation as the baseline. Initially we combined the data sets to compare the doses of BIO 300 evaluated. To analyze the different BIO 300 doses both groups that received 200 mg/kg BIO 300 were combined and compared to the group of animals that received 400 mg/kg BIO 300 (FIG. 8B). BIO 300 dosed at 200 mg/kg significantly reduces the risk of the tumor from doubling (p=0.0036, HR=0.10, 95% CI: 0.1-0.47). The risk of doubling the size of the tumor is reduced by 90% when BIO 300 (200 mg/kg) is combined with radiation treatment. Using the event of tumor doubling, the power in the study was too low to reach statistical significance for the combined BIO 300 400 mg/kg group. Both the calculations of tumor growth delay and the plots analyzing tumor volume growth over time indicate that BIO 300 administered at 400 mg/kg had a therapeutic benefit. Both early and late initiation of BIO 300 dosing is more beneficial than radiation treatment alone. The groups in which dosing was initiated early (200 and 400 mg/kg) were combined and compared to radiation alone. Initiation of BIO 300 dosing early (31 days prior to radiation in this example) was statistically different than radiation alone (p=0.027, HR=0.25, 95% CI: 0.07-0.85). Similarly, the groups in which dosing was initiated late (200 and 400 mg/kg) were combined and compared to radiation alone. Initiation of BIO 300 dosing late (7 days prior to radiation treatment in this study) was statistically different than radiation alone (p=0.027, HR=0.27, 95% CI: 0.09-0.86). Separation of the early and late groups into the different BIO doses was also compared to radiation alone. Initiation of BIO 300 earlier appears to have an added benefit in the 200 mg/kg group (FIG. 8C). By the Cox regression analysis there is no apparent difference between the early and late BIO 300 (400 mg/kg) groups (FIG. 8D). However, tumor growth delay calculations and the plots of tumor growth over time suggest that earlier initiation of dosing for both doses may be more beneficial, i.e. the data is trending toward earlier administration of either dose being more therapeutic. From the analyses completed, BIO 300 dosed at 200 mg/kg had the best efficacy. BIO 300 dosed at 400 mg/kg is trending toward significance. In the absence of adverse BIO 300 side effects, both doses evaluated have shown efficacy.

Tumor Weights:

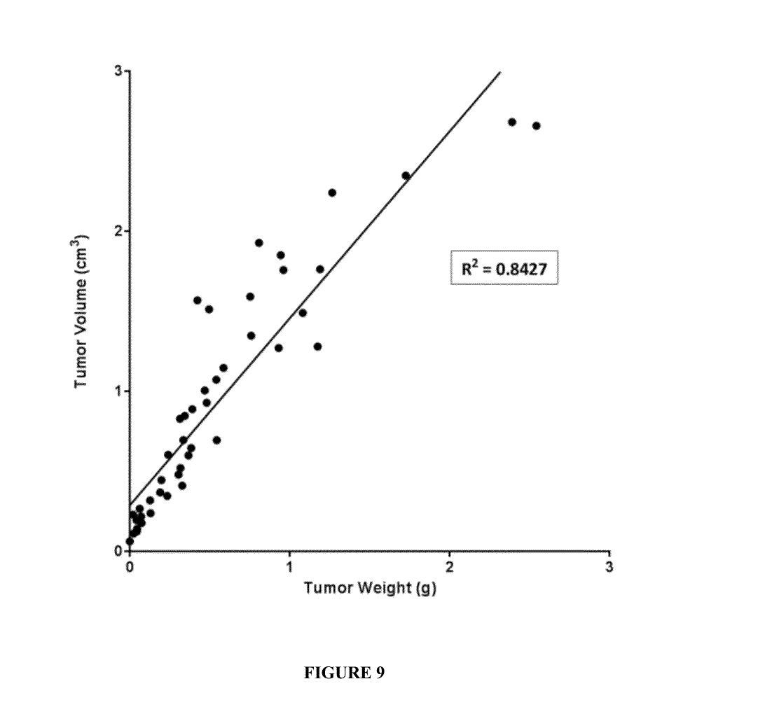

As a means to verify the methods used to calculate tumor volumes we correlated the calculated tumor volumes at the end of the study to the physical weights of the resected and excised tumors. The actual tumor weights were plotted versus the calculated volumes and a regression and correlation analysis was completed. As shown in FIG. 9, the calculated tumor volumes correlated well with the physical weights of the tumors at the time of euthanasia.

Animal Weights:

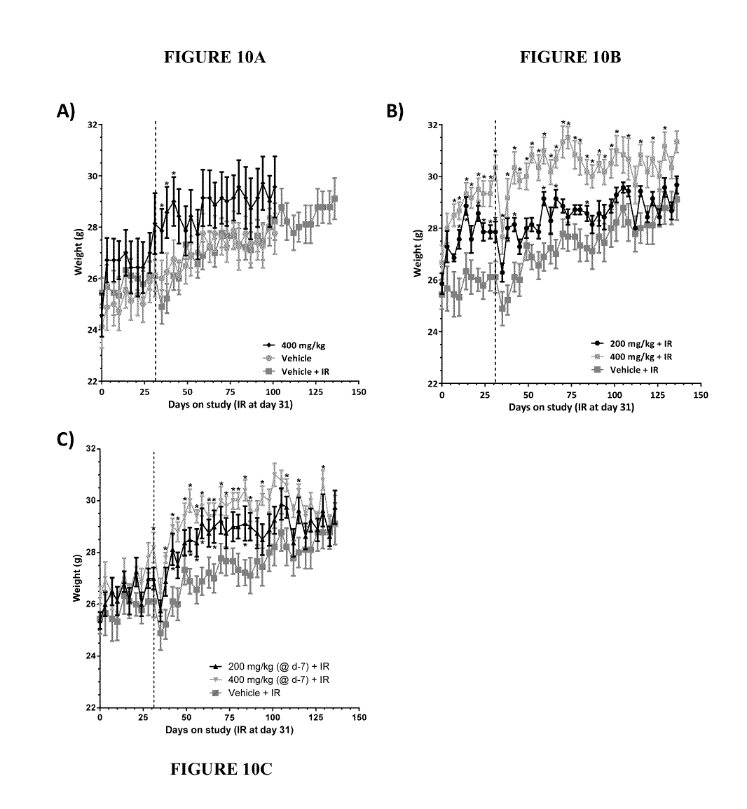

Referring to FIGS. 10A, 10B and 10C, the mean weights of each group throughout the study were plotted and compared to animals that did not receive BIO 300. The data were plotted as the mean±SEM and analyzed from the beginning of the study; the day BIO 300 dosing was initiated. To compare the means on any given day a Student's t-test was performed. The weights of animals that received BIO 300 (with or without radiation) were compared to the animals that received vehicle with radiation. The points marked on the plot (* p<0.05) were statistically different from the mean weight of the animals that received vehicle plus radiation on that given day. To further analyze the data linear mixed statistically modeling was completed to evaluate rate of weight gain between the experimental groups. Analysis of covariance (ANCOVA) was also performed to compare the absolute animal weight pre and post radiation exposure. In this nineteen week study each of the cohorts that received irradiation were exposed to a single 12.5 Gy dose on day 31. For this analysis the timeframe of pre-radiation dosing was defined as days 0-28 and the post radiation timeframe of dosing was defined as day 35 to the end of the study. The pre-radiation weight gain was significantly higher in those groups that received BIO300 (p=0.02, 200 mg/kg; p=0.0018, 400 mg/kg). In addition, the pre-radiation weight was compared in the groups that received BIO 300 (400 mg/kg) from study initiation to the group that received BIO 300 (400 mg/kg) seven days prior to radiation treatment. The animals that received BIO 300 (400 mg/kg) from study initiation had a larger weight gain (p=0.00097). Finally, in those animals that received BIO 300 (400 mg/kg) from study initiation the pre-radiation rate of weight gain was significantly different than the rate in animals that received radiation only (p=0.0509). Taken together, the data indicates that BIO 300 confers an increase in the rate of rate gain resulting in an increased absolute weight that is maintained post radiation exposure. By the end of the study there is no statistical difference between any of the groups, indicating that over an extended period of time BIO 300 does not result in increased body weight.

This maintenance in body weight within the BIO 300 cohorts that received tumor irradiation cannot be explained by tumor weight as those animals had smaller tumors than the group that only received vehicle following radiation. BIO 300 administered prior to and after radiation supports maintenance of body weight. BIO 300 given at 400 mg/kg has a more dramatic effect on body weight. BIO 300 can support a healthy weight during chemoradiation therapy for solid tumor cancers.

Post Mortem Tissue Weights:

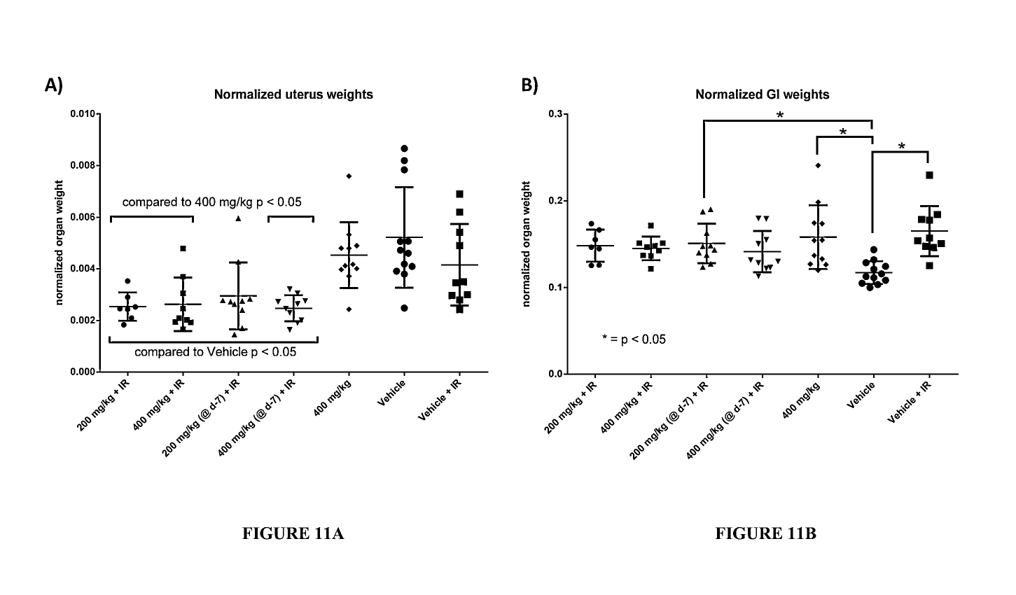

To assess the biological effects of 19 weeks of continuous daily BIO 300 dosing (7 d/wk) the wet tissue weights of the uterus and GI tract were determined following euthanasia. These organ weights were normalized to the weight of the animal at the time of euthanasia as depicted in FIGS. 11A and 11B. To rule out estrogenic effects of prolonged BIO 300 oral administration over the nineteen week study the weights of the uterus were compared between experimental groups. One-way ANOVA followed by Tukey's multiple comparisons post-hoc test was performed to identify statistical differences. All groups that received BIO 300 and radiation had lower uterus weights. Groups that received BIO 300 (400 mg/kg) alone and radiation alone had no effect on uterus weights; the mean weights of these groups were no different than that of the vehicle only group. Because the 400 mg/kg group without radiation does not affect uterus weight and the weight in the BIO 300 with radiation groups is lower than vehicle alone we conclude that in this study BIO 300 does not confer phytoestrogenic side effects.

In this example, the upper GI tract is likely within the path of radiation making this a logical tissue to analyze to further evaluate the protective effects of BIO 300 on normal tissue and also to monitor for detrimental effects from continued BIO 300 administration (safety). We unexpectedly found that the normalized weight of the GI tract was significantly greater in some of the groups compared to the groups that received vehicle alone. Among the groups that are statistically different from the vehicle only group there is no obvious common theme suggesting that multiple biological properties or mechanisms may be contributing to these observations. Assuming that the GI tract was indeed in the path of radiation, the increased weight in the vehicle plus radiation group could have resulted from a radiation-induced inflammatory response and possibly edema. The increase in the other groups may be associated with the BIO 300—mediated increase in body weight observed in both Example 1 and Example 2. However, the GI tract weight was normalized to animal body weight at the time of euthanasia. No obvious abnormalities were observed from the histopathological analysis of the GI tract tissue.

Histopathology (Skin and Terminal Ileum):

Additional tissues were analyzed histologically to further investigate the protective effects of BIO 300 on normal tissues and also determine if continued daily administration of BIO 300 resulted in any adverse effects on the tissues analyzed. The skin near the A549 tumor site and the terminal ileum were analyzed following tissue processing and slide preparation. In this example the tumor was placed in the rear flank, possibly resulting in the terminal ileum positioned in the path of radiation. All tissues were analyzed microscopically following H&E staining. For these analyses 3 skin samples and 3 terminal ileum samples were analyzed from each experimental group. Tissue samples were not analyzed for the BIO 300 groups (200 and 400 mg/kg) in which dosing was initiated seven days prior to tumor irradiation. All tissue samples were prepared, sectioned, and stained with H&E. Tissues were analyzed for gross morphology and H&E sections were evaluated for inflammatory cells, capillary distension, and congestion. A veterinary pathologist scored the severity and distribution of anomalies observed. Severity was scored on a scale from 0-4; (0=unremarkable/normal, 1=minimal, 2=mild, 3=moderate, 4=severe), the distribution was scored as being focal (F), multifocal (M), diffuse (D), or present (P). The conclusions drawn from the histopathological analyses of the different tissues are shown in TABLE SEVEN below.

TABLE SEVEN

| TISSUE | HISTOPATHGLOGY COMMENTS & CONCLUSIONS |
|---|---|
| SKIN | No treatment-related effects were observed in the |
| ADJACENT | microscopic evaluation of sections of skin adjacent |
| TO THE | to the tumor in these mice. |
| TUMOR | |
| TERMINAL | No treatment-related effects were observed in the |
| ILEUM | microscopic evaluation of sections of the terminal |
| | ileum in these mice. Minimal to mild and multifocal |
| | to diffuse amyloid-like material was observed in the |
| | lamina propria of the ileum in all mice in all groups. |
| | The ileum was otherwise normal in appearance. Amyloid |
| | deposition is a common finding in various organs, espe- |
| | cially the ileum, in mice and may be related to age |
| | and/or inflammation. |

Histopathology analysis of the terminal ileum did not identify any adverse tissue responses that were correlated with BIO 300 administration or dose of drug. No adverse effects in the skin above the tumor were observed. Amyloid-like material was observed in the terminal ileum of all mice in all groups. This amyloid deposition was not correlated to BIO 300 administration, but may be related to age and/or inflammation. All together no significant adverse effects of BIO 300 administration were found in any of the tissues analyzed. The data indicates that extended daily BIO 300 oral dosing is safe.

Findings:

BIO 300 at doses up to 400 mg/kg administered daily (7 d/wk) for nineteen weeks does not protect tumors from radiation-induced killing. BIO 300 sensitizes NSCLC tumors in this xenograft model to radiation treatment. BIO 300 plus radiation is more efficacious than radiation alone, increasing the tumor growth delay 31-141%. BIO 300 dosed at 200 mg/kg was more efficacious as a radiosensitizer in the longer duration study. Initiation of BIO 300 dosing either 7 or 31 days prior to radiation treatment conferred an increase in tumor growth delay. Initiation of dosing earlier (31 days prior to radiation) had an increased benefit, suggesting that initiation of BIO 300 dosing as soon as possible after diagnosis may be most beneficial. Administration of BIO 300 promoted maintenance of a stable body weight post radiation exposure. Without intending to be limited thereby, it is believed that this effect results from a more rapid rate of weight gain in this group pre-radiation. The earlier the dosing is initiated, the more pronounced the weight gain. This increase in weight pre-radiation and maintenance of body weight post radiation is temporary as the mean weights of all groups at the end of the study were identical. Histopathological analysis of the skin and the terminal ileum found no adverse treatment related effects from extended BIO 300 dosing for nineteen weeks.

### Example 3

### Post Administration Only

BIO 300 was administered once-daily for fourteen days by oral gavage to separate groups of mice starting at 24, 48, 72, 96 and 120 hours after a single lethal dose of thoracic radiation. Animals were followed for up to 180 days post-radiation for changes in behavior, health, bodyweight, respiratory function, and survival.

Pulmonary Edema:

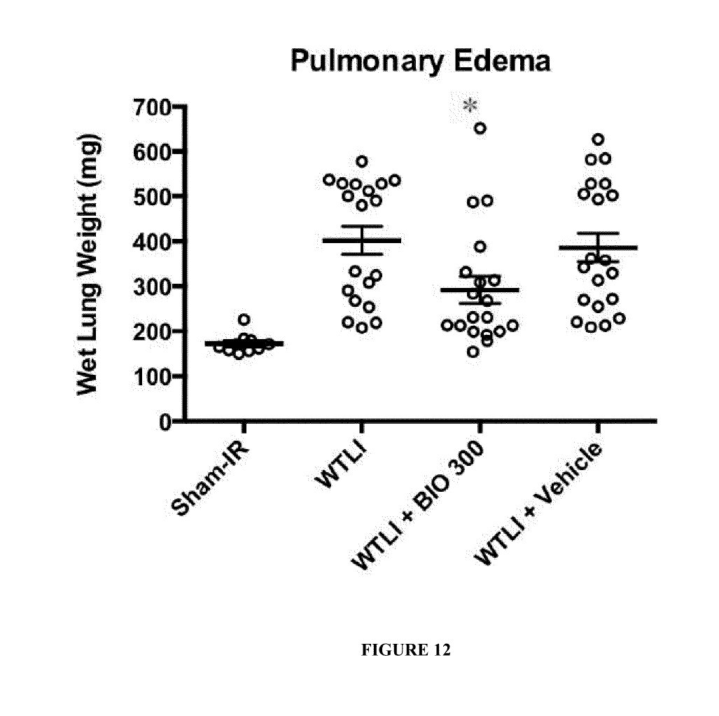

Referring to FIG. 12 and TABLE EIGHT below, giving BIO 300 following radiation significantly reduced the wet lung weights compared to animals receiving radiation alone or those that received the drug vehicle.

TABLE EIGHT

| | | | | | IR + BIO300 | | IR + Vehicle | |
|---|---|---|---|---|---|---|---|---|
| | Sham-IR | | IR Only | | (24 h) | | (24 h) | |
| Mean ± SEM Weight | 172 ± 6.8 | mg | 402 ± 30.9 | mg | 291 ± 29.8 | mg | 386 ± 31.8 | mg |
| Median Weight | 168.0 | mg | 480.0 | mg | 231.0 | mg* | 350.0 | mg |
| 25-75 Percentile | 158-181 | mg | 268-528 | mg | 200-331 | mg | 259-522 | mg |

Most importantly, there was no significant difference in the average lung weight between animals treated with BIO 300 compared to the sham-irradiated animals which suggests radiation has less effect on the lungs of animals treated with BIO 300 at 180 days. Without intending to be limited thereby, this indicates that the potent antioxidant properties of BIO 300 confer protection of lung tissue by suppressing the pro-inflammatory and pro-fibrogenic signaling pathways involved in the development of tissue damage.

Respiratory Function:

Respiratory function was evaluated every two weeks using unrestrained whole body plethysmography. Lung function was assessed based on numerous measured parameters including, respiratory rate, relaxation time, and peak inspiratory and expiratory flow. The observed pathogenic changes in radiation-induced respiratory function were significantly dampened in those animals that received BIO 300 following radiation exposure. Radiation also effected air flow and time to reach peak expiratory flow, both of which were improved in animals treated with BIO 300. The data indicates that in conjunction with radiation exposure BIO 300 administration improves overall lung function.

Fibrosis Score:

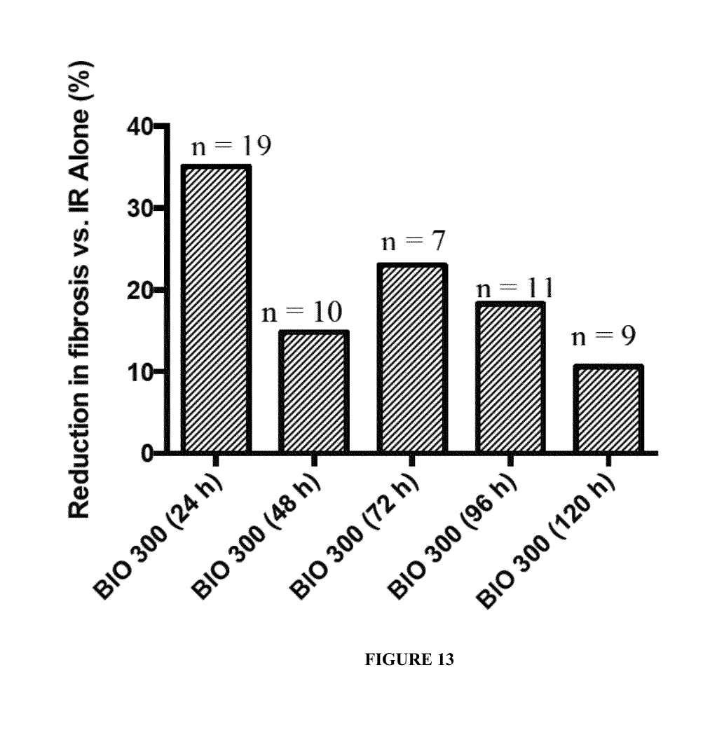

One of the prominent delayed effects resulting from radiation exposure to the lung is an increase in fibrosis which can be observed months after exposure. Referring to FIG. 13 and TABLE NINE below, lung sections stained with Masson's trichrome which stains collagen were analyzed to evaluate the ability of BIO 300 to prevent and/or mitigate the onset of fibrosis. Semi-quantitative assessment of lung fibrosis was determined using a pre-determined numerical scale of 0-8 based on the Ashcroft scoring method, wherein a 1 point change in Ashcroft score is equivalent to 12.5% change in fibrosis. In addition to collagen deposition, histological features such as alveolar wall thickness and fibrotic damage to lung structures were used in the Ashcroft scoring.

TABLE NINE

| | 180 Days | | | vs. IR Only |
|---|---|---|---|---|
| Treatment Group | Male | Female | Average | % Change |
| Irradiation | 6.4 | 6.3 | 6.4 | |
| BIO 300 (24 hrs) | 3.8 | 3.3 | 3.5 | 36.3 |
| BIO 300 (48 hrs) | 5.0 | 5.3 | 5.2 | 15.0 |
| BIO 300 (72 hrs) | 4.9 | 4.1 | 4.5 | 23.8 |
| BIO 300 (96 hrs) | 4.9 | 4.8 | 4.9 | 18.8 |
| BIO 300 (120 hrs) | 5.5 | 5.6 | 5.5 | 11.3 |

Compared to radiation alone, animals treated with BIO 300 had a clear reduction in lung fibrosis. Administration of BIO 300 starting 24 hours post radiation exhibited the greatest reduction in fibrosis (36.3%) compared to BIO 300 treatment starting at 48-120 hours post exposure. Treatment with BIO 300, starting up to 5 days after exposure, demonstrated an 11-36% reduction fibrosis over the irradiation only control. Treatment with BIO 300 at all of the time points evaluated demonstrated an 11-36% reduction in fibrosis over the IR only control.

Histology:

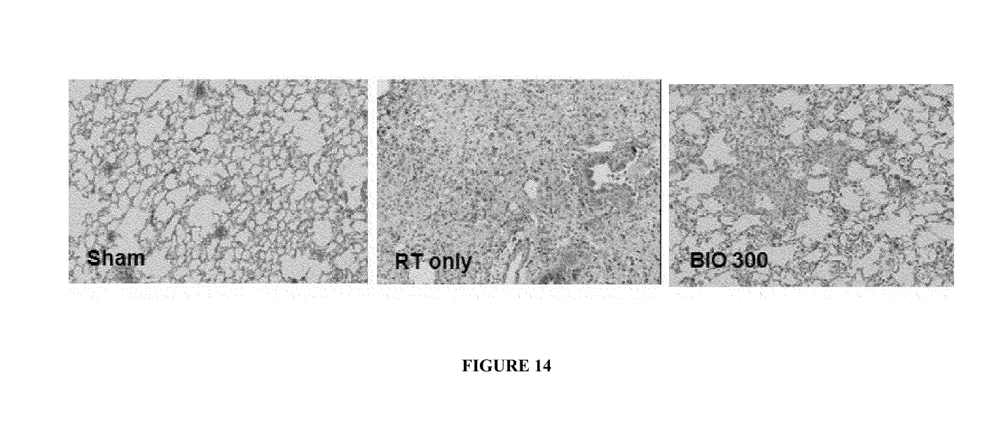

Referring to FIG. 14, at the time of euthanasia H&E stained lung sections were evaluated for inflammatory cells, alveolar capillary distension, and congestions. Upon histopathologic examination, the lungs of animals that received radiation only showed lung damage characteristic of radiation induced injury with diffuse alveolar damage and fibrosis. In contrast, the lungs of animals treated with BIO 300 showed significantly less damage with respect to inflammation and fibrosis and greater overall volume of normal lung. All together the data show a clear difference in the mitigating effect of BIO 300 on radiation-induced lung damage.

The results clearly show that BIO 300 (400 mg/kg/day) protects the normal lung tissue from radiation-induced damage and increases survival following a lethal dose of radiation.

We claim:

1. A treatment regimen for a patient undergoing fractionated radiation therapy for a solid tumor type cancer, comprising administration of a therapeutic amount of genistein to a patient diagnosed with a solid tumor type cancer throughout an administration period that commences at least five days prior to commencement of fractionated cancer radiation therapy and extends until at least the conclusion of fractionated cancer radiation therapy.

2. A treatment regimen for a patient undergoing fractionated radiation therapy for a solid tumor type cancer, effective for attaining an improvement in one or more of cancer cell radiation sensitization, normal cell radioprotection, and selective post-exposure inhibition of cancer cell recover relative to normal cell recovery, the treatment regimen comprising administration of a standard daily therapeutic amount of genistein to a patient diagnosed with a solid tumor type cancer throughout an administration period that commences at least one week prior to commencement of fractionated cancer radiation therapy employing fractionated doses of radiation between about 1.5 and 2.5 Gy per dose, and extends until at least the conclusion of fractionated cancer radiation therapy.

3. The method of claim 1 or 2 wherein the solid tumor type cancer is non-small cell lung cancer.

4. The method of claim 2 wherein administration of a daily therapeutic amount of genistein comprises administration of at least 1 gram per day of genistein.

5. The method of claim 1 wherein the genisitein is administered in daily dosages of at least 1.2 grams.

6. The method of claim 2 wherein administration of a daily therapeutic amount of genistein comprises administration of at least 1.5 grams per day of genistein.

7. The method of claim 1 or 2 wherein the administration period commences at least two weeks prior to commencement of radiation therapy.

8. The method of claim 1 or 2 wherein the administration period commences at least four weeks prior to commencement of radiation therapy.

9. The method of claim 2 wherein a daily maintenance amount of less than 60% the daily standard therapeutic amount is administered during a maintenance period after completion of the administration period.

10. The method of claim 9 wherein the maintenance period is at least one month.

11. The method of claim 9 wherein the maintenance period is at least three months.

12. The method of claim 2 wherein a maintenance amount of between 60% and 100% of the daily standard therapeutic amount is administered during a maintenance period after completion of the administration period, with the maintenance amount administered at a frequency that is one-half or less of the frequency at which the standard daily therapeutic amount was administered.

13. The method of claim 2 wherein a maintenance amount of between 60% and 100% of the daily standard therapeutic amount is administered during a maintenance period after completion of the administration period, with the maintenance amount administered at a frequency that is one-quarter or less of the frequency at which the standard daily therapeutic amount was administered.

14. The method of claim 12 or 13 wherein the maintenance period is at least one month.

15. The method of claim 12 or 13 wherein the maintenance period is at least six months.

16. The method of claim 1 or 2 wherein the genistein is administered in the form of a nanosuspension.

17. The method of claim 1 or 2 wherein administration is effected orally.
